(19) United States
(12) Patent Application Publication
(10) Pub. No.: US 2006/0073110 A1
(43) Pub. Date: Apr. 6, 2006

(54) High DS cationic polygalactomannan for skincare products

(76) Inventor: Jashawant J. Modi, Hockessin, DE (US)

Correspondence Address: Hercules Incorporated;Hercules Plaza, 1313 N. Market Street, Wilmington, DE, 19894-0001, US

(21) Appl. No.: 11/223,525

(22) Filed: Sep. 9, 2005

Related U.S. Application Data
(60) Provisional application No. 60/613,007, filed on Sep. 24, 2004.

(51) Int. Cl. A61K 8/73

(52) U.S. Cl. 424/70.13

(57) ABSTRACT

A skin care composition is provided with a) from about 1 to about 90 wt % of a surfactant, b) at least about 0.05 wt % of a cationic polymer wherein the cationic polymer has a mean average molecular weight (Mw) from about 2,000 to about 10,000,000 Dalton, and the cationic polymer has a cationic degree of substitution (DS) greater than 0.25 to about 3.0, and c) at least one skin care active ingredient, wherein the skin care composition provides at least one of the functions of cleansing, protection, moisturizing, firming, conditioning, occlusive barrier, emolliency, depositing, and anti-wrinkling to the skin.

This application claims the benefit of U.S. Provisional Application No. 60/613,007, filed Sep. 24, 2004.

### FIELD OF THE INVENTION

The present invention relates to a skin care composition and, more particularly, to a skin cleansing, protecting, moisturizing, firming, conditioning, occlusive barrier, and emolliency composition.

### BACKGROUND OF THE INVENTION

In recent years, body washes were not only expected to cleanse the body but also to provide conditioning characteristics. It is well known in the personal care industry to formulate products that provide both cleansing and moisturizing benefits. Many polysaccharides are used as rheology modifiers for skin care products. Cellulose ethers and polygalactomannan and polygalactomannan derivative products are among the best known polysaccharides for use as rheology modifiers.

Cationically derivatized cellulosics and polygalactomannan derivatives are used as a conditioning agent for hair and skin. Cellulosics and polygalactomannan polymers without a cationic group(s) are not used as conditioning agents but rather are used only as a rheology modifier. Most of the commercial cationic polygalactomannans, such as cationic guar products, have cationic degree of substitution (DS) of less than 0.20. Because of manufacturing process related issues, only cationic guar with DS of less than 0.2 is commercially available from raw material suppliers, such as Hercules Incorporated that markets its products under the trademark N-Hance® and Rhodia Corporation that markets its products under the trademarks Jaguar® and Excel®. It is also know in the personal care industry that levels of cationic DS on a polygalactomannan do affect performance of cationic guar in skin care compositions, such as body cleansing and conditioning. The higher the cationic DS, the better the conditioning effect is expected to be at the same use level of the low DS cationic guar. In addition, because of the low DS of the commercial polymers, they are less soluble in water and, therefore, also impact viscosity of the body washes significantly if attempts are made to use higher levels. There are several prior art references that disclose the use of cationic polgalactomannans in personal care products.

US patent Application publication 20030199403 A1 discloses a shampoo containing a cationic guar derivative having a high degree of substitution.

US Patent Application publication 2003/0108507 A1 discloses a hair conditioning shampoo composition of surfactants and cationic polymers.

US Patent Application publication 2004/0157754 A1 discloses a shampoo composition with a conditioning agent and a cationic polymer with a high charge density.

US Patent Application publication 2003/0202952 A1 discloses a shampoo composition with an anti-dandruff agent and a cationic polysaccharide.

US Patent Application publication 2003/0215522 A1 discloses a personal care composition and a zinc anti-dandruff agent and a cationic polymer.

Notwithstanding the prior art, a need still exists in the marketplace for skin care compositions containing cationic polygalactomannans wherein the composition provides better protection to the skin and has significantly improved stability as compared to similar prior art compositions.

### SUMMARY OF THE INVENTION

The present invention is directed to a skin care composition of a) from about 1 to about 90 wt % of a surfactant, b) at least about 0.05 wt % of a cationic polymer wherein i) the cationic polymer has a weight average molecular weight (Mw) from about 2,000 to about 10,000,000 Dalton and ii) the cationic polymer has a degree of substitution (DS) lower limit of 0.25 and has an upper limit of 3.0, and c) at least one skin care active ingredient. The SKin care composition provides protection to the skin. The protection includes cleansing, moisturizing, firming, depositing, conditioning, occlusive barrier, anti-wrinkling, and emolliency to the skin.

Examples of the skin care active ingredient materials are UV absorbers, sun screen agents, moisturizers, humectants, occlusive agents, moisture barriers, lubricants, emollients, anti-aging agents, antistatic agents, secondary conditioners, exfolliants, lustering agents, tanning agents, luminescents, colors, anti-odorizing agents, fragrances, viscosifying agent, salts, lipids, phospholipids, hydrophobic plant extracts, vitamins, silicone oils, silicone derivatives, essential oils, oils, fats, fatty acids, fatty acid esters, fatty alcohols, waxes, polyols, hydrocarbons, and mixture thereof.

### DETAILED DESCRIPTION OF THE INVENTION

It has been surprisingly found that the using of cationic guar polymers having a DS>0.2 provided to skin care products improved protection and greater stability for longer shelf life as compared to similar prior art products. These polymers are significantly lower in aqueous viscosity as compared to cationic guar polymers with DS<0.2, such as N-Hance products (marketed by Aqualon Co.) and Jaguar products (marketed by Rhodia Corp.). Because of the higher DS and lower viscosity that the polymers of the present invention have, they can be used at much higher levels in skin care formulations such as body wash in order to provide skin conditioning and moisturizing. In addition, they provide better salt tolerance than the counterpart guar products with low DS. Other applications for the cationic guar polymers of the present invention are skin and sun care lotions, liquid and bar soaps.

In accordance with the present invention, the cationic guar polymers provide flexibility to work in alkaline pH environment because they have no borax which is commonly used in current commercial cationic guar. In alkaline pH environments, guar polymers cross-link with boron ion and causes it to thicken and even form a gel-like net work which is difficult to dispense through packaging material commonly found for body wash and other skin care products

In accordance with the invention, the polymers that can be used in the invention include cationic galactomannan polymers or cationic derivatized galactomannan polymers having a weight average molecular weight (Mw) having a lower limit of 2,000, preferably 10,000, preferably 50,000, more preferably 100,000, and even more preferably 400,000. The upper limit of the Mw of these polymers are 10,000,000, preferably 5,000,000, more preferably 2,000,000, and even more preferably 1,000,000. Examples of the polygalactomannans of this invention are guar, locust bean, honey locus, and flame tree with guar gum being the preferred source of the polygalactomannan. The preferred polygalactomannan starting material used in this invention is guar flour, guar powder, guar flakes, guar gum, or guar splits which have been derivatized with a cationic substituent.

The preferred polymers of this invention are cationic polygalactomannan polymers. The amount of cationic functionality on the polygalactomannan can be expressed in terms of moles of substituent. The term “degree of substitution” as used in this invention is equivalent to the molar substitution, the average number of moles of functional groups per anhydro sugar unit in the polygalactomannan gum. The cationic functionality can be present on these polymers at a DS lower limit amount of 0.25, preferably about 0.4, and more preferably 0.8. The DS upper limit is normally about 3.0, preferably about 2.0, and more preferably 1.0.

The cationic functionality of the polygalactomannan or derivatized polygalactomannan can be added to them by several methods. For example, the starting material can be reacted for a sufficient time and at a sufficient temperature with tertiary amino compound or quaternary ammonium compound containing groups capable of reacting with the reactive hydrogen ions present on the polygalactomannan or derivatized polygalactomannan in order to add the cationic functionality to the starting material. The sufficient time depends on the ingredients in the reaction mass and the temperature under which the reaction is taking place.

The cationizing agent of the present invention is defined as a compound which, by substitution reaction with the hydroxy groups of the polygalactomannan can make the product electrically positive, and there is no limitation to its types. Tertiary amino compounds or various quaternary ammonium compounds containing groups capable of reacting with reactive hydrogen present on the polysaccharide, can be used, such as 2-dialkylaminoethyl chloride and quaternary ammonium compounds such as 3-chloro-2-hydroxypropyltrimethylammonium chloride, and 2,3-epoxy-propyltrimethylammonium chloride. Preferred examples include glycidyltrialkylammonium salts and 3-halo-2-hydroxypropyltrialkylammonium salts such as glycidyltrimethylammonium chloride, glycidyltriethylammonium chloride, gylcidyltripropylammonium chloride, glycidylethyldimethylammonium chloride, glycidyldiethylmethylammonium chloride, and their corresponding bromides and iodides; 3-chloro-2-hydroxypropyltrimethylammonium chloride, 3-chloro-2-hydroxypropyltriethylammonium chloride, 3-chloro-2-hydroxypropyltripropylammonium chloride, 3-chloro-2-hydroxypropylethyldimethylammonium chloride, and their corresponding bromides and iodides; and quaternary ammonium compounds such as halides of imidazoline ring containing compounds.

Other derivatization of the cationic polygalactomannan with nonionic substituents, i.e., hydroxyalkyl wherein the alkyl represents a straight or branched hydrocarbon moiety having 1 to 6 carbon atoms (e.g., hydroxyethyl, hydroxypropyl, hydroxybutyl) or anionic substituents, such as carboxymethyl groups are optional. These optional substituents are linked to the polygalactomannan molecule by the reaction of the polygalactomannan molecule with reagents such as (1) alkylene oxides (e.g., ethylene oxide, propylene oxide, butylene oxide) to obtain hydroxyethyl groups, hydroxypropyl groups, or hydroxybutyl groups, or with (2) chloromethyl acetic acid to obtain a carboxymethyl group on the polygalactomannan. This reaction can take place when the polygalactomannan is in the “split”, “flour” or any other physical form. The process for preparing derivatized polygalactomannan is well known in the art.

In accordance with the present invention, surfactants are an important ingredient in skin care formulations and can be used either alone of in combination with other type of surfactants. The role of these surface active agents is to reduce surface tension when dissolved in water or water solution, or to reduce interfacial tension between two liquids, or between a liquid and a solid. These characteristics are often used in providing in removal of undesirable material from substrate such as skin or textile substrates. Examples of these surfactants include anionic, nonionic, cationic, zwitterionic, amphoteric or mixtures thereof type of surfactants. It is quite common to use mixtures of these surfactants in skin care products. The surfactant can be insoluble (or soluble) in the present invention and (when used) is present in the composition in the amount of from 1.0 to 90% by weight of the composition.

Anionic surfactants include alkyl and alkyl ether sulfates. Specific examples of alkyl ether sulfates which can be used in the present invention are sodium coconut alkyl trimethylene glycol ether sulfate; sodium tallow alkyl trimethylene glycol ether sulfate; sodium tallow alkyl hexaoxyethylene sulfate; sodium tallow alkyl diethylene glycol ether sulfate; and sodium tallow alkyl sulfate. Other example of anionic surfactants are sulfonates, sulfosuccinates, sacosinates, carboxylates, and isethionates.

Nonionic surfactants can be broadly defined as compounds containing a hydrophobic moiety and a nonionic hydrophilic moiety. Examples of the hydrophobic moiety can be alkyl, alkyl aromatic, dialkyl siloxane, polyoxyalkylene, and fluoro-substituted alkyls. Examples of hydrophilic moieties are polyoxyalkylenes, phosphine oxides, sulfoxides, amine oxides, and amides. Other examples of nonionic surfactants include alkyl polysaccharides such as alkyl polysaccharides.

Cationic surfactants useful in the compositions of the present invention contain amino or quaternary ammonium hydrophilic moieties which are positively charged when dissolved in an aqueous composition of the present invention.

Zwitterionic surfactants are exemplified by those which can be broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic water-solubilizing group, e.g., carboxy, sulfonate, sulfate, phosphate, or phosphonate.

Examples of amphoteric surfactants which can be used in the compositions of the present invention are those which are broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group, e.g., carboxy, sulfonate, sulfate, phosphate, or phosphonate.

In accordance with the present invention, the skin care active ingredient must provide some benefit to the user's body. Skin care products includes, body wash, shower cream, shower gels, liquid soaps, bar soaps, skin lotions, skin creams, after shower lotions, after cleansing lotions, shave products, after shave products, and deodorant products, antiperspirant products, skin cleansing wipes, skin cooling wipes, skin conditioning wipes, skin drug delivery products, insect repellent products, and sun care products. Examples of substances that may suitably be included in the skin care products as active ingredients according to the present invention are as follows:

1) Perfumes, which give rise to an olfactory response in the form of a fragrance and deodorant perfumes which in addition to providing a fragrance response can also reduce body malodor;

2) Skin coolants, such as menthol, menthyl acetate, menthyl pyrrolidone carboxylate N-ethyl-p-menthane-3-carboxamide and other derivatives of menthol, which give rise to a tactile response in the form of a cooling sensation on the skin;

3) Emollients, such as isopropylmyristate, silicone materials, mineral oils and vegetable oils which give rise to a tactile response in the form of an increase in skin lubricity;

4) Deodorants other than perfumes, whose function is to reduce the level of or eliminate micro flora at the skin surface, especially those responsible for the development of body malodor. Precursors of deodorants other than perfume can also be used;

5) Antiperspirant actives, whose function is to reduce or eliminate the appearance of perspiration at the skin surface;

6) Moisturizing agents, that keeps the skin moist by either adding moisture or preventing from evaporating from the skin;

7) Cleansing agents, that removes dirt and oil from the skin;

8) Shaving products, such as creams, gels and lotions and razor blade lubricating strips;

9) Tissue paper products, such as moisturizing or cooling or cleansing tissues;

10) Beauty aids, such as foundation powders, lipsticks, and eye care;

11) Textile products, such as moisturizing or cleansing wipes;

12) Nail care products;

13) Abrasives which acts as exfoliate;

14) Skin bleaching and lightening agents—(e.g., hydroquinone, kojic acid, arbutin, ascorbic acid and derivatives thereof, (e.g., magnesium ascorbyl phosphate, sodium ascorbyl phosphate, and ascorbyl glucosamine) and extracts (e.g., mulberry extract, placental extract) as well as titanium dioxide and zinc oxide.). These skin lightening agents are used in preferred concentrations of from about 0.1% to about 10%, more preferably from about 0.2% to about 5%, more preferably from about 0.5% to about 2%, by weight of the composition;

15) Skin soothing and/or healing agents-include panthenoic acid derivatives, (e.g., panthenol, dexpanthenol and ethyl panthenol), aloe vera, pantothenic acid and its derivatives, allantoin, bisabolol, and dipotassium glycyrrhizinate), retinoids, (e.g. retinol palmitate), tocopheryl nicotinate, skin treating agents, vitamins and derivatives thereof. These actives are preferably used in concentrations from about 0.1% to about 30%, more preferably from about 0.5% to about 20%, still more preferably from about 0.5% to about 10%, by weight of the composition;

16) Desquamation Actives—preferred concentrations of which range from about 0.1% to about 10%, Desquamation actives enhance the skin appearance benefits of the present invention. For example, the desquamation actives tend to improve the texture of the skin (e.g., smoothness). One desquamation system that is suitable for use herein contains sulfhydryl compounds and zwitterionic surfactants;

17) Anti-Acne Actives—preferred concentrations of which range from about 0.01% to about 50%, more preferably from about 1% to about 20%, by weight of the composition. Non-limiting examples of anti-acne actives suitable for use herein include resorcinol, sulfur, salicylic acid, benzoyl peroxide, erythromycin, zinc, and other similar materials;

18) Anti-Wrinkle Actives/Anti-Atrophy Actives—including sulfur-containing D and L amino acids and their derivatives and salts, particularly the N-acetyl derivatives, a preferred example of which is N-acetyl-L-cysteine; thiols, e.g. ethane thiol; hydroxy acids (e.g., alpha-hydroxy acids such as lactic acid and glycolic acid or beta-hydroxy acids such as salicylic acid and salicylic acid derivatives such as the octanoyl derivative), phytic acid, lipoic acid; lysophosphatidic acid, and skin peel agents (e.g., phenol and the like). Also suitable is niacinamide. Hydroxy acids as skin benefit agents herein include salicylic acid and salicylic acid derivatives, preferred concentrations of which range from about 0.01% to about 50%, more preferably from about 0.1% to about 10%, even more preferably from about 0.5% to about 2%, by weight of the composition;

19) Anti-Oxidants/Radical Scavengers—The skin benefit agent for use herein can also include anti-oxidants or radical scavengers, preferred concentrations of which range from about 0.1% to about 10%, more preferably from about 1% to about 5%, by weight of the composition. Examples of anti-oxidants or radical scavengers for use herein include ascorbic acid and its salts, ascorbyl esters of fatty acids, ascorbic acid derivatives (e.g., magnesium ascorbyl phosphate, sodium ascorbyl phosphate, ascorbyl sorbate), tocopherol, tocopherol acetate, other esters of tocopherol, butylated hydroxy benzoic acids and their salts, 6-hydroxy-2,5,7,8-tetramethylchroman-2-carboxylic acid (commercially available under the tradename Trolox.RTM.), gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, lycine pidolate, arginine pilolate, nordihydroguaiaretic acid, bioflavonoids, curcumin, lysine, methionine, proline, superoxide dismutase, silymarin, tea extracts, grape skin/seed extracts, melanin, and rosemary extracts may be used;

20) Chelators or Chelating agent—refers to those skin benefit agents capable of removing a metal ion from a system by forming a complex so that the metal ion cannot readily participate in or catalyze chemical reactions. The chelating agents as skin benefit agents for use herein are preferably formulated at concentrations ranging from about 0.1% to about 10%, more preferably from about 1% to about 5%, by weight of the composition. Preferred chelating agents for use in the active phase of the compositions of the present invention include furildioxime, furilmonoxime, and derivatives thereof;

21) Flavonoids agent-includes flavonoid compounds suitable for use on the hair or skin, preferred concentrations of which range from about 0.01% to about 20%, more preferably from about 0.1% to about 10%, more preferably from about 0.5% to about 5%, by weight of the composition. Examples of flavonoids compounds suitable for use as skin benefit agents include flavanones such as unsubstituted flavanones, mono-substituted flavanones, and mixtures thereof; chalcones selected from unsubstituted chalcones, mono-substituted chalcones, di-substituted chalcones, tri-substituted chalcones, and mixtures thereof; flavones selected from unsubstituted flavones, mono-substituted flavones, di-substituted flavones, and mixtures thereof; one or more isoflavones; coumarins selected from unsubstituted coumarins, mono-substituted coumarins, di-substituted coumarins, and mixtures thereof; chromones selected from unsubstituted chromones, mono-substituted chromones, di-substituted chromones, and mixtures thereof; one or more dicoumarols; one or more chromanones; one or more chromanols; isomers (e.g., cis/trans isomers) thereof; and mixtures thereof. By the term “substituted” as used herein means flavonoids wherein one or more hydrogen atom of the flavonoid has been independently replaced with hydroxyl, C1-C8 alkyl, C1-C4 alkoxyl, O-glycoside, and the like or a mixture of these substituents;

22) Anti-Inflammatory Agents—preferred concentrations of which range from about 0.1% to about 10%, more preferably from about 0.5% to about 5%, by weight of the composition;

Examples of steroidal anti-inflammatory agents suitable for use herein include corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof may be used. The preferred steroidal anti-inflammatory for use is hydrocortisone.

Nonsteroidal anti-inflammatory agents are also suitable for use herein as skin benefit agents in the active phase of the compositions;

23) Anti-Cellulite Agents—include xanthine compounds such as caffeine, theophylline, theobromine, aminophylline, and combinations thereof;

24) Topical Anesthetics—include benzocaine, lidocaine, bupivacaine, chlorprocaine, dibucaine, etidocaine, mepivacaine, tetracaine, dyclonine, hexylcaine, procaine, ketamine, pramoxine, phenol, pharmaceutically acceptable salts thereof, and combinations thereof;

25) Tanning Actives—preferred concentrations of which range from about 0.1% to about 20% by weight of the composition. Non-limiting examples of such tanning agents include dihydroxyacetone, which is also known as DHA or 1,3-dihydroxy-2-propanone;

26) Antimicrobial Actives—The skin benefit agent for use in compositions of the present invention may include antimicrobial actives, preferred concentrations of which range from about 0.001% to about 10%, more preferably from about 0.01% to about 5%, and still more preferably from about 0.05% to about 2%, by weight of the compositions;

Examples of antimicrobial actives for use herein includes .beta.-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, phenoxy propanol, phenoxyisopropanol, doxycycline, capreomycin, chlorhexidine, chlortetracycline, oxytetracycline, clindamycin, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, ketaconazole, amanfadine hydrochloride, amanfadine sulfate, octopirox, parachlorometa xylenol, nystatin, tolnaftate, zinc pyrithione, clotrimazole, and combinations thereof;

27) Sunscreen Actives—which may be either organic or inorganic sunscreen actives. Among the inorganic sunscreens useful hererin are metallic oxides such as titanium dioxide having an average primary particle size of from about 15 nm to about 100 nm, zinc oxide having an average primary particle size of from about 15 nm to about 150 nm, zirconium oxide having an average primary particle size of from about 15 nm to about 150 nm, iron oxide having an average primary particle size of from about 15 nm to about 500 nm, and mixtures thereof;

Examples of organic sunscreen actives include p-aminobenzoic acid, its salts and its derivatives (ethyl, isobutyl, glyceryl esters; p-dimethylaminobenzoic acid); anthranilates (i.e., o-amino-benzoates; methyl, menthyl, phenyl, benzyl, phenylethyl, linalyl, terpinyl, and cyclohexenyl esters); salicylates (amyl, phenyl, octyl, benzyl, menthyl, glyceryl, and di-pro-pyleneglycol esters); cinnamic acid derivatives (menthyl and benzyl esters, a-phenyl cinnamonitrile; butyl cinnamoyl pyruvate); dihydroxycinnamic acid derivatives (umbelliferone, methylumbelliferone, methylaceto-umbelliferone); trihydroxy-cinnamic acid derivatives (esculetin, methylesculetin, daphnetin, and the glucosides, esculin and daphnin); hydrocarbons (diphenylbutadiene, stilbene); dibenzalacetone and benzalacetophenone; naphtholsulfonates (sodium salts of 2-naphthol-3,6-disulfonic and of 2-naphthol-6,8-disulfonic acids); di-hydroxynaphthoic acid and its salts; o- and p-hydroxybiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); quinoline derivatives (8-hydroxyquinoline salts, 2-phenylquinoline); hydroxy- or methoxy-substituted benzophenones; uric and violuric acids; tannic acid and its derivatives (e.g., hexaethylether); (butyl carbotol) (6-propyl piperonyl) ether; hydroquinone; benzophenones (oxybenzene, sulisobenzone, dioxybenzone, benzoresorcinol, 2,2′,4,4′-tetrahydroxybenzophenone, 2,2′-dihydroxy-4,4′-dimethoxybenzophenone, octabenzone; 4-isopropyldibenzoylmethane; butylmethoxydibenzoylmethane; etocrylene; octocrylene; [3-(4′-methylbenzylidene bornan-2-one), terephthalylidene dicamphor sulfonic acid and 4-isopropyl-di-benzoylmethane. Among these sunscreens, preferred are 2-ethylhexyl-p-methoxycinnamate (commercially available as PARSOL MCX), 4,4′-t-butyl methoxydibenzoyl-methane (commercially available as PARSOL 1789), 2-hydroxy-4-methoxybenzophenone, octyldimethyl-p-aminobenzoic acid, digalloyltrioleate, 2,2-dihydroxy-4-methoxybenzophenone, ethyl-4-(bis(hydroxypropyl))aminobenzoate, 2-ethylhexyl-2-cyano-3,3-diphenylacrylate, 2-ethylhexyl-salicylate, glyceryl-p-aminobenzoate, 3,3,5-tri-methylcyclohexylsalicylate, methylanthranilate, p-dimethyl-aminobenzoic acid or aminobenzoate, 2-ethylhexyl-p-dimethyl-amino-benzoate, 2-phenylbenzimidazole-5-sulfonic acid, 2-(p-dimethylaminophenyl)-5-sulfonicbenzoxazoic acid, octocrylene and combinations thereof; and

28) Visual Skin Enhancers—include ingredients that mask the appearance of any number of skin imperfections such as age spot, fine lines, wrinkles, blemishes etc., including but not limited to titanium dioxide, zinc oxide and iron oxides. Also suitable for use herein are organic particulates that diffuse light when deposited on the skin. Preferred concentrations of these ingredients range from about 0.001% to about 10%, more preferably from about 0.01% to about 5%, and still more preferably from about 0.05% to about 2%, by weight of the compositions.

The above list of skin care active ingredients are only examples and are not a complete lists of active ingredients that can be used. Other ingredients that are used in these types of products are well known in the industry. In addition to the above ingredients conventionally used, the composition according to the present invention can optionally also include ingredients such as a colorant, preservative, nutritional supplements, activity enhancer, emulsifiers, functional polymers, viscosifying agents (such as NaCl, NH4Cl, KCl, Na₂SO₄,fatty alcohols, fatty acid esters, fatty acid amides, fatty alcohol polyethyleneglycol ethers, sorbitol polyethyleneglycol ethers, cocamide monoethanolamide, cocamide diethanolamide, cocamidopropyl betaine, clays, silicas, cellulosic polymers, and xanthan), suspending agents (such as clays, silica, and xanthan), stabilizers, alcohols having 1-6 carbons, fats or fatty compounds, zinc pyrithione, silicone material, hydrocarbon polymer, oils, medicaments, flavors, fragrances, rejuvenating reagents, and mixtures thereof.

In accordance with the present invention, examples of functional polymers that can be used in blends with the cationic polygalactomannan or derivatives thereof of this invention include water-soluble polymers such as anionic, hydrophobically-modified, and amphoteric acrylic acid copolymers, vinylpyrrolidone homopolymers; cationic, hydrophobically-modified, and amphoteric vinylpyrrolidone copolymers; nonionic, cationic, anionic, and amphoteric cellulosic polymers such as hydroxyethylcellulose, hydroxypropylcellulose, carboxymethylcellulose, hydroxypropylmethylcellulose, cationic hydroxyethylcellulose, cationic carboxymethylhydroxyethylcellulose, and cationic hydroxypropylcellulose; acrylamide homopolymers and cationic, amphoteric, and hydrophobically-modified acrylamide copolymers, polyethylene glycol polymers and copolymers, hydrophobically-modified polyethers, hydrophobically-modified polyetheracetals, hydrophobically-modified polyols and polyetherurethanes and other polymers referred to as associative polymers, hydrophobically-modified cellulosic polymers, polyethyleneoxide-propylene oxide copolymers, and nonionic, anionic, hydrophobically-modified, amphoteric, and cationic polysaccharides such as xanthan, chitosan, carboxymethyl guar, alginates, hydroxypropyl guar, carboxymethyl guar hydroxypropyltrimethylammonium chloride, guar hydroxypropyltrimethylammonium chloride, hydroxypropyl guar hydroxypropyltrimethylammonium chloride.

In accordance with the invention, the silicone materials which can be used are, in particular, polyorganosiloxanes that are insoluble in the composition and can be in the form of polymers, oligomers, oils, waxes, resins, or gums.

The organopolysiloxanes are defined in greater detail in Walter Noll's “Chemistry and Technology of Silicones” (1968) Academic Press. They can be volatile or non volatile.

If volatile, the silicones are more particularly chosen from those having a boiling point of between 60° C. and 260° C., and even more particularly from:

(i) cyclic silicones containing from 3 to 7 and preferably from 4 to 5 silicon atoms. These are, for example, octamethylcyclotetrasiloxane sold in particular under the name “Volatile Silicone 7207” by Union Carbide or “Silbione 70045 V 2” by Rhone Poulenc, decamethyl cyclopentasiloxane sold under the name “Volatile Silicone 7158” by Union Carbide, and “Silbione 70045 V 5” by Rhone Poulenc, and mixtures thereof.

Mention may also be made of mixtures of cyclic silicones with organosilicone compounds, such as the mixture of octamethylcyclotetrasiloxane and tetratrimethylsilylpentaerythritol (50/50) and the mixture of octamethylcyclotetrasiloxane and oxy 1,1′bis(2,2,2′,2′,3,3′ hexatrimethylsilyloxy) neopentane;

(ii) linear volatile silicones having 2 to 9 silicon atoms and having a viscosity of less than or equal to 5×10⁻⁶ m2/s at 25° C. An example is decamethyltetrasiloxane sold in particular under the name “SH 200” by Toray Silicone company. Silicones belonging to this category are also described in the article published in Cosmetics and Toiletries, Vol. 91, Jan. 76, pp. 27 32, Todd & Byers “Volatile Silicone Fluids for Cosmetics”.

Non volatile silicones, and more particularly polyarylsiloxanes, polyalkylsiloxanes, polyalkylarylsiloxanes, silicone gums and resins, polyorganosiloxanes modified with organofunctional groups, and mixtures thereof, are preferably used.

In accordance with the invention, the silicone polymers and resins which can be used are, in particular, polydiorganosiloxanes having high number-average molecular weights of between 200,000 and 1,000,000, used alone or as a mixture in a solvent. This solvent can be chosen from volatile silicones, polydimethylsiloxane (PDMS) oils, polyphenylmethylsiloxane (PPMS) oils, isoparaffins, polyisobutylenes, methylene chloride, pentane, dodecane and tridecane, or mixtures thereof.

Examples of these silicone polymers and resins are as follows:
Polydimethylsiloxane,
polydimethylsiloxanes/methylvinylsiloxane gums,
polydimethylsiloxane/diphenylmethylsiloxane,
polydimethylsiloxane/phenylmethylsiloxane, and
polydimethylsiloxane/diphenylsiloxanemethylvinylsiloxane.

Products which can be used more particularly in accordance with the invention are mixtures such as:
(a) mixtures formed from a polydimethylsiloxane hydroxylated at the end of the chain (referred to as dimethiconol according to the nomenclature in the CTFA dictionary) and from a cyclic polydimethylsiloxane (referred to as cyclomethicone according to the nomenclature in the CTFA dictionary), such as the product Q2 1401 sold by the Dow Corning Company;
(b) mixtures formed from a polydimethylsiloxane gum with a cyclic silicone, such as the product SF 1214 Silicone Fluid from the company General Electric Company; this product is an SF 30 gum corresponding to a dimethicone, having a number average molecular weight of 500,000, dissolved in SF 1202 Silicone Fluid oil corresponding to decamethylcyclopentasiloxane; and
(c) mixtures formed of two PDMSs of different viscosities, and more particularly of a PDMS gum and a PDMS oil, such as the product SF 1236 from the General Electric Company. The product SF 1236 is a mixture of a gum SE 30 defined above, having a viscosity of 20 m2/s, and an oil SF 96, with a viscosity of 5×10⁻⁶ m2/s. This product preferably contains 15% SE 30 gum and 85% SF 96 oil.

These silicone materials function as conditioning agents for skin surfaces. Other types of conditioning agents include oils, waxes, hydrocarbon oils, such as mineral oil and fatty acid ester of glycerol, and panthenol and its derivatives, such as panthenyl ethyl ether, panthenyl hydroxypropyl steardimonium chloride, and pantothenic acid.

Oils include hydrocarbon oils and waxes, silicones, fatty acid derivatives, cholesterol, cholesterol derivatives, diglycerides, triglycerides, vegetable oils, vegetable oil derivatives, acetoglyceride esters, alkyl esters, alkenyl esters, lanolin and its derivatives, wax esters, beeswax derivatives, sterols and phospholipids, and combinations thereof.

Examples of hydrocarbon oils and waxes suitable for use herein include petrolatum, mineral oil, micro-crystalline waxes, polyalkenes, paraffins, cerasin, ozokerite, polyethylene, perhydrosqualene, poly alpha olefins, hydrogenated polyisobutenes and combinations thereof.

Examples of silicone oils suitable for use herein include dimethicone copolyol, dimethylpolysiloxane, diethylpolysiloxane, mixed C1-C30 alkyl polysiloxanes, phenyl dimethicone, dimethiconol, and combinations thereof. Preferred are non-volatile silicones selected from dimethicone, dimethiconol, mixed C1-C30 alkyl polysiloxane, and combinations thereof. Nonlimiting examples of silicone oils useful herein are described in U.S. Pat. No. 5,011,681 (Ciotti et al.).

Examples of diglycerides and triglycerides suitable for use herein include castor oil, soy bean oil, derivatized soybean oils such as maleated soy bean oil, safflower oil, cotton seed oil, corn oil, walnut oil, peanut oil, olive oil, cod liver oil, almond oil, avocado oil, palm oil and sesame oil, vegetable oils, sunflower seed oil, and vegetable oil derivatives; coconut oil and derivatized coconut oil, cottonseed oil and derivatized cottonseed oil, jojoba oil, cocoa butter, and combinations thereof. In addition any of the above oils that have been partially or fully hydrogenated are also suitable.

Examples of acetoglyceride esters suitable for use herein include acetylated monoglycerides.

Examples of alkyl esters suitable for use herein include isopropyl esters of fatty acids and long chain esters of long chain fatty acids, e.g. SEFA (sucrose esters of fatty acids). Lauryl pyrolidone carboxylic acid, pentaerthritol esters, aromatic mono, di or triesters, cetyl ricinoleate, non-limiting examples of which include isopropyl palmitate, isopropyl myristate, cetyl riconoleate and stearyl riconoleate. Other examples are: hexyl laurate, isohexyl laurate, myristyl myristate, isohexyl palmitate, decyl oleate, isodecyl oleate, hexadecyl stearate, decyl stearate, isopropyl isostearate, diisopropyl adipate, diisohexyl adipate, dihexyldecyl adipate, diisopropyl sebacate, acyl isononanoate lauryl lactate, myristyl lactate, cetyl lactate, and combinations thereof.

Examples of alkenyl esters suitable for use herein include oleyl myristate, oleyl stearate, oleyl oleate, and combinations thereof.

Examples of lanolin and lanolin derivatives suitable for use herein include lanolin, lanolin oil, lanolin wax, lanolin alcohols, lanolin fatty acids, isopropyl lanolate, acetylated lanolin, acetylated lanolin alcohols, lanolin alcohol linoleate, lanolin alcohol riconoleate, hydroxylated lanolin, hydrogenated lanolin and combinations thereof.

Still other suitable oils include milk triglycerides (e.g., hydroxylated milk glyceride) and polyol fatty acid polyesters.

Still other suitable oils include wax esters, non-limiting examples of which include beeswax and beeswax derivatives, spermaceti, myristyl myristate, stearyl stearate, and combinations thereof. Also useful are vegetable waxes such as carnauba and candelilla waxes; sterols such as cholesterol, cholesterol fatty acid esters; and phospholipids such as lecithin and derivatives, sphingo lipids, ceramides, glycosphingo lipids, and combinations thereof.

The suitable stabilizers include Pemulen TR-1 (Acrylates/C10-30 Alkyl Acrylate Crosspolymer-Noveon), Pemulen TR-2 (Acrylates/C10-30 Alkyl Acrylate Crosspolymer-Noveon), ETD 2020 (Acrylates/C10-30 Alkyl Acrylate Crosspolymer-Noveon), Carbopol 1382 (Acrylates/C10-30 Alkyl Acrylate Crosspolymer-Noveon), Natrosol CS Plus 330, 430, Polysurf 67 (Cetyl Hydroxyethyl Cellulose-Hercules), Aculyn 22 (Acrylates/Steareth-20 Methacrylate Copolymer-Rohm&Haas) Aculyn 25 (Acrylates/Laureth-25 Methacrylate copolymer-Rohm&Haas), Aculyn 28 (Acrylates/Beheneth-25 Methacrylate copolymer-Rohm&Haas), Aculyn 46 (Peg-150/Stearyl Alcohol/SMDI copolymer-Rohm&Haas) Stabylen 30 (AcrylatesNinyl Isodecanoate-3V), Structure 2001 (Acrylates/Steareth-20 Itaconate copolymer-National Starch), Structure 3001 (Acrylates/Ceteth-20 Itaconate copolymer-National Starch), Structure Plus (Acrylates/Aminoacrylates/C10-30 Alkyl Peg 20 Itaconate copolymer-National Starch, Quatrisoft LM-200 (Polyquaternium-24), the metal oxides of titanium, zinc, iron, zirconium, silicon, manganese, aluminium and cerium, polycarbonates, polyethers, polyethylenes, polypropylenes, polyvinyl chloride, polystyrene, polyamides, polyacrylates, cyclodextrins and mixtures thereof.

Cyclodextrins are solubilized, water-soluble, uncomplexed cyclodextrins. As used herein, the term “cyclodextrin” includes any of the known cyclodextrins such as unsubstituted cyclodextrins containing from six to twelve glucose units, especially, alpha-cyclodextrin, beta-cyclodextrin, gamma-cyclodextrin and/or their derivatives and/or mixtures thereof. Examples of preferred water-soluble cyclodextrin derivatives suitable for use herein are hydroxypropyl alpha-cyclodextrin, methylated alpha-cyclodextrin, methylated beta-cyclodextrin, hydroxyethyl beta-cyclodextrin, and hydroxypropyl beta-cyclodextrin. Cyclodextrins particularly preferred for use herein are alpha cyclodextron, beta cyclodextron, hydroxypropyl alpha cyclodextrin, hydroxypropyl beta cyclodextrin, and a mixture thereof.

For a more detailed understanding of the invention, reference can be made to the following examples which are intended as further illustration of the invention and are not to be construed in a limiting sense. All parts and percentages are by weight unless stated otherwise.

### EXAMPLE 1

### Body Wash—Sodium Luareth Sulfate/Cocamideamidopropyl betaine/Sodium Chloride with N-Hance® 3215 and with AQU D3799 (Table 1 & 2)

This Example is to illustrate the invention and compare it with a prior art commercial cationic polymer, N-Hance® 3215 product, that has a cationic DS of about 0.2 and a weight average molecular weight (Mw) of 1,350,000 via size exclusion chromatography. A cationic guar product of the present invention, referred to as AQU D3799, has cationic DS of 0.63 and a weight average Molecular weight of 1,120,000.

A stock solution of the body wash was made with a 10% “hole” in it to add salt thickener at a later stage has the following formulation:
35.31 g Deionized water
0.50 g Cationic guar N-Hance® 3215
43.64 g Sodium Laureth Sulfate (SLES)
10.00 g Cocamidopropyl betaine (CAPB)
00.50 g DMDM Hydantoin

The N-Hance 3215 product was added to the water while mixing. Next, 5% citric acid solution was added to N-Hance product and water mixture in order to lower the pH to about 6.0. This mixture was mixed for an hour. The SLES was added slowly to this mixture while mixing. The new mixture was continued being mixed until the body wash mixture looked homogeneous. Next, the CAPB was added while mixing. Again, the mixture was allowed to mix until homogenous. Next, DMDM Hydantoin was added and mixed for about 10 minutes. The pH of the body wash was then adjusted to between 6 to 6.5 with citric acid or sodium hydroxide solutions.

To 90.0 grams of this body wash, 25% sodium chloride solution was added to achieve desired salt concentration in the final body wash formulation. For salt level above 2.5% active, dry salt was added to the body wash. The same procedure was followed for the polymer of this invention, AQU D3799.

The body wash viscosity was measured with Brookfield LVT viscometer. Viscosity was measured at 25° C. Product stability was visually observed for homogeneity, insoluble gels or phase separation. Body wash clarity was visually rated as being “clear”, “very slightly hazy,” “slightly hazy”, “moderately hazy”, and “very hazy”, “considerable hazy”, “sever hazy” and “opaque”. This type of rating is common in the Personal Care Industry for comparative expression of product clarity study. Clarity also was measured at 600 nm using a Spectrophotometer, Cary 5E UV-VIS-NIR, available from Varian Instruments, Inc., or equivalent. The clarity measurements at 600 nm wavelength are reported as % T value in the tables. The higher the number, the clearer is the solution.

In Tables 1 and 2, the two cationic guars were compared for their compatibility in sodium laureth sulfate, cocamidopropyl betaine and sodium chloride based body wash systems. Sodium chloride is a salt found in body wash systems as a rheology modifier. With the N-Hance® 3215 product, a commercially available cationic guar product, the body wash system was unstable without the salt and with a very low salt level. In addition, as the salt level was increased, the body wash system became unstable which was reflected by poor clarity. Clarity was measured by measuring % T (transmittance at 600 nm). At 3.0% salts levels insoluble gels were observed. With the AQU D3799 polymer of this invention, the body wash system was unstable in absence of sodium chloride or at very low level of salt. However, it did not show instability with increase in salt level as did the commercial N-Hance 3215 products.

A blind panel test was used to determine whether an individual can aesthetically feel differences in body wash formulations prepared with the current commercial N-Hance® 3215 product versus with the polymer of this invention, AQU D3799. In the blind test a test member was asked to wash her hands first with about 40° C. plain water. Next, 2.0 ml of body wash was dispensed in the palm of a test member's hands. The test member was asked to wash all sides of both hands with the body wash for 30 seconds. Next, the panel members were asked to wash their hands thoroughly with plain 40° C. water for 30 seconds. Hands were then pad dried with paper towels. During this cycle the test member was asked to comment on lather properties, lather volume, ease of lather, ease of rinsing and skin after feel. According to the test member, the body wash with polymer of the invention gave very rich lather, was less slippery, and was more conditioning as compared to the body wash formulated with the commercial N-Hance® 3215 product.

TABLE 1

| | X33646- | | | | | | | | | | |
|---|---|---|---|---|---|---|---|---|---|---|---|
| | 50-1A | 50-1B | 50-1C | 50-1D | 50-1E | 50-1F | 50-1G | 50-1H | 50-1I | 50-1J | 50-1K |
| | Wt | Wt | Wt | Wt | Wt | Wt | Wt | Wt | Wt | Wt | Wt |
| Ingredients | (gr.) | (gr.) | (gr.) | (gr.) | (gr.) | (gr.) | (gr.) | (gr.) | (gr.) | (gr.) | (gr.) |
| Deionized | 35.31 | 35.31 | 35.31 | 35.31 | 35.31 | 35.31 | 35.31 | 35.31 | 35.31 | 35.31 | 35.31 |
| water | | | | | | | | | | | |
| N- | 0.50 | 0.50 | 0.50 | 0.50 | 0.50 | 0.50 | 0.50 | 0.50 | 0.50 | 0.50 | 0.50 |
| Hance ® | | | | | | | | | | | |
| 3215 | | | | | | | | | | | |
| Sodium | 43.63 | 43.63 | 43.63 | 43.63 | 43.63 | 43.63 | 43.63 | 43.63 | 43.63 | 43.63 | 43.63 |
| Laureth | | | | | | | | | | | |
| Sulfate | | | | | | | | | | | |
| Cocamidopropyl | 10.00 | 10.00 | 10.00 | 10.00 | 10.00 | 10.00 | 10.00 | 10.00 | 10.00 | 10.00 | 10.00 |
| betaine | | | | | | | | | | | |
| DMDM | 00.5 | 00.5 | 00.5 | 00.5 | 00.5 | 00.5 | 00.5 | 00.5 | 00.5 | 00.5 | 00.5 |
| Hydantoin | | | | | | | | | | | |
| Sodium | 00.0 | 0.25 | 0.5 | 0.75 | 1.0 | 1.25 | 1.75 | 2.5 | 3.0 | 3.5 | 5.0 |
| Chloride | | | | | | | | | | | |
| Deionized | 10.00 | 9.00 | 8.00 | 7.00 | 6.00 | 5.00 | 3.00 | 0.00 | 0.00 | 0.00 | 0.00 |
| Water | | | | | | | | | | | |
| Total | 100 | 100 | 100 | 100 | 100 | 100 | 100 | 100 | 100 | 100 | 100 |
| pH | 6.3 | 6.6 | 6.5 | 6.7 | 6.6 | 6.6 | 6.5 | 6.6 | 6.7 | 6.5 | 6.9 |
| Viscosity | 420 | 460 | 520 | 615 | 612 | 792 | 1612 | 6440 | 15220 | 15300 | 17580 |
| (cps), | | | | | | | | | | | |
| 30 rpm | | | | | | | | | | | |
| Comment | Separation | Slight | Very Hazy | Very Hazy | Very Hazy | Very Hazy | Very Hazy | Sever | Gels | Gels | Severe |
| | | settling | | | | | | Hazy | | | Hazy Very |
| | | | | | | | | | | | Thixotropic |
| % T | Not | 53 | 51 | 50 | 47 | 39 | 15 | 5 | Gels | Gels | 6.8 |
| | Determined | | | | | | | | | | |

Ingredients:
(1) Cationic guar, 100% active, N-Hance® 3215, Cationic DS 0.19, Molecular weight 1,350,000, Hercules Inc. Wilmington, Del., USA
(2) Sodium Laureth sulfate, 27.5% active, Rhodapex® ES-STD, Rhodia, Cranbury, N.J., USA
(3) Cocamidopropyl betaine, 30% active, Amphosol® CA, Stepan Company, Northfield, Ill., USA
(4) DMDM Hydantoin, Glydant®, Lonza Inc. Fair Lawn, N.J., USA

(5) NaCl was added as 25% solution up to 2.5% active. Rest was added dry.

TABLE 2

| | X33646- | | | | | | | | | | |
|---|---|---|---|---|---|---|---|---|---|---|---|
| | 50-2A | 50-2B | 50-2C | 50-2D | 50-2E | 50-2F | 50-2G | 50-2H | 50-2I | 50-2J | 50-2K |
| | Wt | Wt | Wt | Wt | Wt | Wt | Wt | Wt | Wt | Wt | Wt |
| Ingredients | (gr.) | (gr.) | (gr.) | (gr.) | (gr.) | (gr.) | (gr.) | (gr.) | (gr.) | (gr.) | (gr.) |
| Deionized | 35.31 | 35.31 | 35.31 | 35.31 | 35.31 | 35.31 | 35.31 | 35.31 | 35.31 | 35.31 | 35.31 |
| water | | | | | | | | | | | |
| AQU | 0.50 | 0.50 | 0.50 | 0.50 | 0.50 | 0.50 | 0.50 | 0.50 | 0.50 | 0.50 | 0.50 |
| D3799 | | | | | | | | | | | |
| Sodium | 43.63 | 43.63 | 43.63 | 43.63 | 43.63 | 43.63 | 43.63 | 43.63 | 43.63 | 43.63 | 43.63 |
| Laureth | | | | | | | | | | | |
| Sulfate | | | | | | | | | | | |
| Cocamidopropyl | 10.00 | 10.00 | 10.00 | 10.00 | 10.00 | 10.00 | 10.00 | 10.00 | 10.00 | 10.00 | 10.00 |
| betaine | | | | | | | | | | | |
| DMDM | 00.5 | 00.5 | 00.5 | 00.5 | 00.5 | 00.5 | 00.5 | 00.5 | 00.5 | 00.5 | 00.5 |
| Hydantoin | | | | | | | | | | | |
| Sodium | 00.0 | 0.25 | 0.5 | 0.75 | 1.0 | 1.25 | 1.75 | 2.5 | 3.0 | 3.5 | 5.0 |
| Chloride | | | | | | | | | | | |
| Deionized | 10.00 | 9.00 | 8.00 | 7.00 | 6.00 | 5.00 | 3.00 | 0.00 | 0.00 | 0.00 | 0.00 |
| Water | | | | | | | | | | | |
| Total | 100 | 100 | 100 | 100 | 100 | 100 | 100 | 100 | 100 | 100 | 100 |
| batch | | | | | | | | | | | |
| pH | 6.3 | 6.4 | 6.4 | 6.3 | 6.3 | 6.4 | 6.4 | 6.4 | 6.4 | 6.6 | 6.5 |
| Viscosity | 81 | 146 | 252 | 281 | 406 | 591 | 1480 | 7480 | 15820 | 15360 | 19400 |
| (cps), | | | | | | | | | | | |
| 30 rpm | | | | | | | | | | | |
| Comment | Separation | Separation | Slight | Slight | Hazy | Hazy | Haze | Slight | Smooth. | Smooth. | Sever |
| | | | sediment | sediment | | | | Hazy | No Gels. | No Gels | Haze, No |
| | | | | | | | | | Slight hazy | | gel |
| % T | | | 49 | 55 | 55 | 57 | 65 | 86 | 92 | 88 | 6 |

(1) Cationic Guar—AQU D3799, cationic DS of 0.63 and the weight average Molecular weight of 1,120,000. Aqualon, Wilmington, Del., USA

### EXAMPLE 2

### Body Wash—Ammonium Laureth Sulfate/Cocamidamidopropyl betaine/Sodium Chloride with N-HanCe® 3215 and with AQU D3799 (Tables 3 & 4)

This Example illustrates the performance comparison of commercial N-HanCe® 3215 cationic guar to product of this invention, AQU D3799. N-Hance® 3215 product has cationic DS of about 0.19 and the weight average molecular weight of 1,350,000 per size exclusion chromatography. The AQU D3799 product has a cationic DS of 0.63 and a weight average molecular weight of 1,120,000.

A stock solution body wash was made with a 10% “hole” in it to add salt thickener at a later stage as follows:
37.81 g Deionized water
0.50 g cationic guar N-Hance® 3215
42.14 g Ammonium Laureth Sulfate (ALES)
9.00 g Cocamidopropyl betaine (CAPB)
00.50 g DMDM Hydantoin

N-Hance® 3215 product was added to the water while mixing. Next, 5% citric acid solution was added to lower pH to about 6.0 and then the mixture was mixed for an hour. ALES was added slowly while mixing and the mixture was continued mixing until the body wash looked homogeneous. Next, CAPB was added while mixing. Again, the mixture was allowed to mix until homogenous. Next, DMDM Hydantoin was added and mixed for about 10 minutes. The pH of the body wash was adjusted between 6 to 6.5 with citric acid or sodium hydroxide solutions.

To 90.0 grams of this body wash, 25% sodium chloride solution was added to achieve the desired salt concentration in final body wash formulation. For salt level above 2.5% active, dry salt was added to body wash. For polymer of this invention, AQU D3799, the same procedure was used In the ammonium laureth sulfate, cocamidopropyl betaine based body wash, the product of this invention AQU D3799 mentioned previously showed better product stability than the commercial product N-Hance® 3215 available from Aqualon. See Tables 3 and 4.

A blind test was carried out to determine if a test subject could detect differences in body wash formulations prepared with the current commercial N-Hance® 3215 product versus with AQU D3799 of this invention. In the blind test the test subject was asked first to wash her hands with about plain 40° C. water. Next, 2.00 ml of body wash was dispensed in the palm of the test subject's hands and the test subject was asked to wash all sides of both hands with the body wash for 30 seconds. Next, the test subject was asked to wash her hands thoroughly under 40° C. water for 30 seconds. The hands were then pad dried with paper towel. During this cycle the test subject was asked to comment on lather properties, lather volume, ease of lather, ease of rinsing and skin after feel. According to the test subject body wash with polymer of the invention gave better lather, richer lather, less slippery, good conditioning as compared to body wash formulated with the commercial N-Hance® 3215 product.

TABLE 3

| | X33646- | | | | | | | | | | |
|---|---|---|---|---|---|---|---|---|---|---|---|
| | 50-3A | 50-3B | 50-3C | 50-3D | 50-3E | 50-3F | 50-3G | 50-3H | 50-3I | 50-3J | 50-3K |
| | Wt | Wt | Wt | Wt | Wt | Wt | Wt | Wt | Wt | Wt | Wt |
| Ingredients | (gr.) | (gr.) | (gr.) | (gr.) | (gr.) | (gr.) | (gr.) | (gr.) | (gr.) | (gr.) | (gr.) |
| Deionized | 37.81 | 37.81 | 37.81 | 37.81 | 37.81 | 37.81 | 37.81 | 37.81 | 37.81 | 37.81 | 37.81 |
| water | | | | | | | | | | | |
| N-Hance ® | 0.50 | 0.50 | 0.50 | 0.50 | 0.50 | 0.50 | 0.50 | 0.50 | 0.50 | 0.50 | 0.50 |
| 3215 | | | | | | | | | | | |
| Ammonium | 42.14 | 42.14 | 42.14 | 42.14 | 42.14 | 42.14 | 42.14 | 42.14 | 42.14 | 42.14 | 42.14 |
| Laureth | | | | | | | | | | | |
| Sulfate | | | | | | | | | | | |
| Cocamidopropyl | 9.00 | 9.00 | 9.00 | 9.00 | 9.00 | 9.00 | 9.00 | 9.00 | 9.00 | 9.00 | 9.00 |
| betaine | | | | | | | | | | | |
| DMDM | 00.5 | 00.5 | 00.5 | 00.5 | 00.5 | 00.5 | 00.5 | 00.5 | 00.5 | 00.5 | 00.5 |
| Hydantoin | | | | | | | | | | | |
| Sodium | 00.0 | 0.25 | 0.5 | 0.75 | 1.0 | 1.25 | 1.75 | 2.5 | 3.0 | 3.5 | 5.0 |
| Chloride | | | | | | | | | | | |
| Deionized | 10.00 | 9.00 | 8.00 | 7.00 | 6.00 | 5.00 | 3.00 | 0.00 | 0.00 | 0.00 | 0.00 |
| Water | | | | | | | | | | | |
| Total | 100 | 100 | 100 | 100 | 100 | 100 | 100 | 100 | 100 | 100 | 100 |
| batch | | | | | | | | | | | |
| pH | 5.5 | 5.6 | 5.5 | 5.5 | 5.5 | 5.4 | 5.4 | 5.4 | 5.3 | 5.3 | 5.2 |
| Viscosity | 532 | 577 | 650 | 720 | 875 | 1095 | 1420 | 2372 | 3780 | Separation | Separation |
| (cps), | | | | | | | | | | | |
| 30 rpm | | | | | | | | | | | |
| Comment | Slight | Hazy | Hazy | Hazy | Hazy | Hazy | Hazy | Hazy | Hazy | Gels | Gels |
| | sediment | | | | | | | | | | |
| % T | 58 | 49 | 48 | 50 | 48 | 46 | 34 | 8 | 9 | | |

Ingredients
(1) Cationic guar, 100% active, N-Hance® 3215, Molecular weight 1,350,000, Cationic DS 0.19 Hercules Inc. Wilmington, Del., USA
(2) Aluminum Laureth sulfate, 28% active, Steol® CA330, Stepan Company, Northfield, Ill., USA
(3) Cocamidopropyl betaine, 30% active, Amphosol® CA, Stepan Company, Northfield, Ill., USA

(4) DMDM Hydantoin, Glydant®, Lonza Inc. Fair Lawn, N.J., USA

TABLE 4

| | X33646- | | | | | | | | | | |
|---|---|---|---|---|---|---|---|---|---|---|---|
| | 50-4A | 50-4B | 50-4C | 50-4D | 50-4E | 50-4F | 50-4G | 50-4H | 50-4I | 50-4J | 50-4K |
| | Wt | Wt | Wt | Wt | Wt | Wt | Wt | Wt | Wt | Wt | Wt |
| Ingredients | (gr.) | (gr.) | (gr.) | (gr.) | (gr.) | (gr.) | (gr.) | (gr.) | (gr.) | (gr.) | (gr.) |
| Deionized | 37.81 | 37.81 | 37.81 | 37.81 | 37.81 | 37.81 | 37.81 | 37.81 | 37.81 | 37.81 | 37.81 |
| water | | | | | | | | | | | |
| AQU | 0.50 | 0.50 | 0.50 | 0.50 | 0.50 | 0.50 | 0.50 | 0.50 | 0.50 | 0.50 | 0.50 |
| D3799 | | | | | | | | | | | |
| Ammonium | 42.14 | 42.14 | 42.14 | 42.14 | 42.14 | 42.14 | 42.14 | 42.14 | 42.14 | 42.14 | 42.14 |
| Laureth | | | | | | | | | | | |
| Sulfate | | | | | | | | | | | |
| Cocamidopropyl | 9.00 | 9.00 | 9.00 | 9.00 | 9.00 | 9.00 | 9.00 | 9.00 | 9.00 | 9.00 | 9.00 |
| betaine | | | | | | | | | | | |
| DMDM | 00.5 | 00.5 | 00.5 | 00.5 | 00.5 | 00.5 | 00.5 | 00.5 | 00.5 | 00.5 | 00.5 |
| Hydantoin | | | | | | | | | | | |
| Sodium | 00.0 | 0.25 | 0.5 | 0.75 | 1.0 | 1.25 | 1.75 | 2.5 | 3.0 | 3.5 | 5.0 |
| Chloride | | | | | | | | | | | |
| Deionized | 10.00 | 9.00 | 8.00 | 7.00 | 6.00 | 5.00 | 3.00 | 0.00 | 0.00 | 0.00 | 0.00 |
| Water | | | | | | | | | | | |
| Total batch | 100 | 100 | 100 | 100 | 100 | 100 | 100 | 100 | 100 | 100 | 100 |
| pH | 5.5 | 5.6 | 5.6 | 5.6 | 5.5 | 5.5 | 5.5 | 5.4 | 5.4 | 5.3 | 5.2 |
| Viscosity | 108 | 200 | 310 | 380 | 460 | 630 | 890 | 2100 | 3520 | 6000 | 15300 |
| (cps), | | | | | | | | | | | |
| 30 rpm | | | | | | | | | | | |
| Comment | separation | Separation | Hazy | Hazy | Hazy | Hazy | Hazy | Slight | Slight | Clear | Hazy |
| | | | | | | | | Hazy | Hazier | | |
| % T | | | 46 | 53 | 55 | 56 | 62 | 86 | 93 | 94 | 14 |

Cationic guar, AQU D3799, Cationic DS 0.63, Molecular weight 1,120,000. Aqualon, Wilmington, DE, USA

### EXAMPLE 3

### Body Wash Ammonium Laureth Sulfate/Cocamidopropyl betaine/Sodium Chloride with, ADPP 5040, with ADPP5199, with AQU D3798 and with GPX 247. (Tables 5 & 6)

Body wash stock solution was made with 10% “hole” in it to add salt thickener at a later stage as follows:
37.81 g Deionized water
0.50 g GPX 247
42.14 g Ammonium Laureth Sulfate (ALES)
9.00 g Cocamidopropyl betaine (CAPB)
00.50 g DMDM Hydantoin

GPX 247 was added to water while mixing. Next, 5% citric acid solution was added to lower pH to about 6.0 and then mixed for an hour. ALES was added slowly while mixing and the mixture was continued mixing until the body wash looked homogeneous. Next, the CAPB was added while mixing. Again, the mixtured was allowed to mix until homogenous. Next, DMDM Hydantoin was added and mixed for about 10 minutes. The pH of the Body wash was adjusted between 6 to 6.5 with citric acid or sodium hydroxide solutions.

To 90.0 grams of this body wash 25% sodium chloride solution was added to achieve desired salt concentration in the final body wash formulation. For salt level above 2.5% active, dry salt was added to body wash. For polymer of this invention, ADPP5199, ADPP5040 and AQU D3798, the same procedure was used.

In Tables 5 and 6, performance data for body wash formulated with the three cationic guar polymers of this invention are reported. They are compared for their stability performance to commercially available cationic guar polymer GPX 247 based body wash. The body wash prepared with commercial polymer GPX 247 were unstable in presence of sodium chloride. Compatibility of the polymer of this invention as well as viscosity of body wash improves in the presence of salt.

TABLE 5

| | X33646- | | | | | | | | | | |
|---|---|---|---|---|---|---|---|---|---|---|---|
| | 81-1A | 81-1B | 81-1C | 81-1D | 81-1E | 81-2A | 81-2B | 81-2C | 81-2D | 81-2E | |
| | Wt | Wt | Wt | Wt | Wt | Wt | Wt | Wt | Wt | Wt | Wt |
| Ingredients | (gr.) | (gr.) | (gr.) | (gr.) | (gr.) | (gr.) | (gr.) | (gr.) | (gr.) | (gr.) | (gr.) |
| Deionized | 37.81 | 37.81 | 37.81 | 37.81 | 37.81 | 37.81 | 37.81 | 37.81 | 37.81 | 37.81 | |
| water | | | | | | | | | | | |
| ADPP 5040 | 0.50 | 0.50 | 0.50 | 0.50 | 0.50 | | | | | | |
| ADPP 5191 | | | | | | 0.50 | 0.50 | 0.50 | 0.50 | 0.50 | |
| Ammonium | 42.14 | 42.14 | 42.14 | 42.14 | 42.14 | 42.14 | 42.14 | 42.14 | 42.14 | 42.14 | |
| Laureth | | | | | | | | | | | |
| Sulfate | | | | | | | | | | | |
| Cocamidopropyl | 9.00 | 9.00 | 9.00 | 9.00 | 9.00 | 9.00 | 9.00 | 9.00 | 9.00 | 9.00 | |
| betaine | | | | | | | | | | | |
| DMDM | 00.5 | 00.5 | 00.5 | 00.5 | 00.5 | 00.5 | 00.5 | 00.5 | 00.5 | 00.5 | |
| Hydantoin | | | | | | | | | | | |
| Sodium | 00.0 | 2.0 | 3.5 | 5.0 | 6.37 | 0.00 | 2.00 | 3.5 | 5.0 | 6.3 | |
| Chloride | | | | | | | | | | | |
| Deionized | 10.00 | 8.00 | 7.5 | 7.5 | 7.5 | 10.00 | 8.00 | 7.5 | 7.5 | 6.66 | |
| Water | | | | | | | | | | | |
| Total batch | 100 | 100 | 100 | 100 | 100 | 100 | 100 | 100 | 100 | 100 | |
| pH | 5.6 | 5.4 | 5.3 | 5.2 | 5.1 | 5.8 | 5.5 | 5.3 | 5.2 | 5.2 | |
| Viscosity | Separated | Separated | 3724 | 10320 | 16020 | 88 | 780 | 3840 | 10380 | 18600 | |
| (cps), 30 rpm | | | | | | | | | | | |
| Comment | Separated | Separated | Slight | clear | Clear | Very | Very slight | | | | |
| | | | haze | | | Hazy | hazy | | | | |
| % T | | | 71.5 | 96 | 96.5 | 60.1 | 92.6 | 21.0 | 16.2 | 19.2 | |

TABLE 6

| | X33646- | | | | | | | | | | |
|---|---|---|---|---|---|---|---|---|---|---|---|
| | 81-3A | 81-3B | 81-3C | 81-3D | 81-3E | 81-4A | 81-4B | 81-4C | 81-4D | 81-4E | |
| | Wt | Wt | Wt | Wt | Wt | Wt | Wt | Wt | Wt | Wt | Wt |
| Ingredients | (gr.) | (gr.) | (gr.) | (gr.) | (gr.) | (gr.) | (gr.) | (gr.) | (gr.) | (gr.) | (gr.) |
| Deionized water | 37.81 | 37.81 | 37.81 | 37.81 | 37.81 | 37.81 | 37.81 | 37.81 | 37.81 | 37.81 | |
| AQU D3798 | 0.50 | 0.50 | 0.50 | 0.50 | 0.50 | | | | | | |
| GPX 247 | | | | | | 0.50 | 0.50 | 0.50 | 0.50 | 0.50 | |
| Ammonium | 42.14 | 42.14 | 42.14 | 42.14 | 42.14 | 42.14 | 42.14 | 42.14 | 42.14 | 42.14 | |
| Laureth Sulfate | | | | | | | | | | | |
| Cocamidopropyl | 9.00 | 9.00 | 9.00 | 9.00 | 9.00 | 9.00 | 9.00 | 9.00 | 9.00 | 9.00 | |
| betaine | | | | | | | | | | | |
| DMDM | 00.5 | 00.5 | 00.5 | 00.5 | 00.5 | 00.5 | 00.5 | 00.5 | 00.5 | 00.5 | |
| Hydantoin | | | | | | | | | | | |
| Sodium | 00.0 | 2.0 | 3.5 | 5.0 | 6.2 | 0.00 | 2.00 | 3.5 | 5.0 | 6.2 | |
| Chloride | | | | | | | | | | | |
| Deionized | 10.00 | 8.00 | 7.5 | 7.5 | 6.8 | 10.00 | 8.00 | 7.5 | 7.5 | 6.8 | |
| Water | | | | | | | | | | | |
| Total batch | 100 | 100 | 100 | 100 | 100 | 100 | 100 | 100 | 100 | 100 | |
| pH | 5.6 | 5.4 | 5.3 | 5.2 | 5.1 | 5.8 | 5.5 | 5.3 | 5.2 | 5.2 | |
| Viscosity (cps), | 11..0 | 294 | 2612 | 8240 | 15120 | Separated | Separated | Separated | Separated | Separated | |
| 30 rpm | | | | | | | | | | | |
| Comment | Separated | Separated | Slight haze | Moderate | Very Hazy | Separated | Separated | Separated | Separated | Separated | |
| | | | | haze | | | | | | | |
| % T | | | 92 | 75.5 | 9.3 | | | | | | |

(1) Aluminum Laureth sulfate, 28% active, Steol® CA330, Stepan Company, Northfield, Ill., USA
(2) Cocamidopropyl betaine, 30% active, Amphosol® CA, Stepan Company, Northfield, Ill., USA
(3) DMDM Hydantoin, Glydant®, Lonza Inc. Fair Lawn, N.J., USA
(4) ADPP5040 is an experimental cationic guar from Hercules Inc. It has Molecular weight of about 1,900,000 and cationic DS of about 1.3
(5) ADPP51991 is an experimental cationic guar from Hercules Inc. It has Molecular weight of about 1,600,000 and cationic DS of about 0.33.
(6) AQU D3798 is an experimental cationic guar from Hercules Inc. It has Molecular weight of about 600,000 and cationic DS of about 0.7
(7) GPX 247, is a commercial cationic guar from Hercules Inc. It has Molecular weight of 760,000 and cationic DS of 0.11

### EXAMPLE 4

### Body Wash—Sodium Laureth Sulfate/Cocamidopropyl betaine/Sodium Chloride with. ADPP 5040, with ADPP5199, with AQU D3798 and with GPX 247. (Table 7 & 8)

Stock solution of body wash was prepared with 20% “hole” in it to add salt thickener at later stage as follows:
25.31 g Deionized water
0.50 g Cationic guar of this invention ADPP5040
43.64 g Sodium Laureth Sulfate (SLES)
10.00 g Cocamidopropyl betaine (CAPB)
00.50 g DMDM Hydantoin

AQU D3798 cationic guar was added to water while mixing. Next 5% citric acid solution was added to lower pH to about 6.0 and then continued to mix for an hour. SLES was added slowly while mixing and then was allowed to mix until homogeneous. Next, CAPB was added while mixing. Again, the mixture was allowed to mix until homogenous. Next, DMDM Hydantoin was added and mixed for about 10 minutes. The pH was adjusted with citric acid or sodium hydroxide solution to between 6 to 6.5.

To 80.0 grams of this body wash 25% sodium chloride solution was added to achieve desired salt concentration in final body wash formulation. Water was added as necessary to bring total weight to 100 grams

In Tables 7 and 8, performance data for the body wash with three cationic guar polymers of this inventions were compared for their stability performance to commercially available cationic guar polymer GPX 247 based body wash. Body Wash with commercial polymer GPX 247 were either not as stable or not as clear as those made with cationic guar of this invention (i.e., ADPP 5040, ADPP 5199, and AQUD 3798).

TABLE 7

| | X33272 | | | | | | | | | | | |
|---|---|---|---|---|---|---|---|---|---|---|---|---|
| | 35A | 35B | 35C | 35D | 35E | 35F | 36A | 36B | 36C | 36D | 36E | 36F |
| | Wt | Wt | Wt | Wt | Wt | Wt | Wt | Wt | Wt | Wt | Wt | Wt |
| Ingredients | (gr.) | (gr.) | (gr.) | (gr.) | (gr.) | (gr.) | (gr.) | (gr.) | (gr.) | (gr.) | (gr.) | (gr) |
| Deionized | 25.31 | 25.31 | 25.31 | 25.31 | 25.31 | 25.31 | 25.31 | 25.31 | 25.31 | 25.31 | 25.31 | 25.31 |
| water | | | | | | | | | | | | |
| ADPP5040 | 0.50 | 0.50 | 0.50 | 0.50 | 0.50 | 0.50 | | | | | | |
| ADPP5199 | | | | | | | 0.50 | 0.50 | 0.50 | 0.50 | 0.50 | 0.50 |
| Sodium | 43.64 | 43.64 | 43.64 | 43.64 | 43.64 | 43.64 | 43.64 | 43.64 | 43.64 | 43.64 | 43.64 | 43.64 |
| Laureth | | | | | | | | | | | | |
| Sulfate | | | | | | | | | | | | |
| Cocamidopropyl | 10.00 | 10.00 | 10.00 | 10.00 | 10.00 | 10.00 | 10.00 | 10.00 | 10.00 | 10.00 | 10.00 | 10.00 |
| betaine | | | | | | | | | | | | |
| DMDM | 00.5 | 00.5 | 00.5 | 00.5 | 00.5 | 00.5 | 00.5 | 00.5 | 00.5 | 00.5 | 00.5 | 00.5 |
| Hydantoin | | | | | | | | | | | | |
| Sodium | 00.00 | 1.00 | 2.00 | 3.5 | 5.00 | 7.00 | 00.0 | 1.00 | 2.00 | 3.5 | 5.0 | 7.0 |
| Chloride | | | | | | | | | | | | |
| Deionized | 20.00 | 19.00 | 18.00 | 16.5 | 15.00 | 15.00 | 20.0 | 19.0 | 18.0 | 16.5 | 15.0 | 15.0 |
| Water | | | | | | | | | | | | |
| Total | 100 | 100 | 100 | 100 | 100 | 100 | 100 | 100 | 100 | 100 | 100 | 100 |
| batch | | | | | | | | | | | | |
| pH | | | | 6.2 | 6.00 | 6.3 | 6.3 | 6.3 | 6.35 | 6.2 | 6.2 | 6.3 |
| Viscosity | Sep- | Sep- | Sep- | 14740 | 76400 | 70000 | 62 | 247 | 2276 | 14960 | 18840 | 72900 |
| (cps), | arated | arated | arated | | | | | | | | | |
| 30 rpm | | | | | | | | | | | | |
| Comment | | | | | | | Consider- | Very | Slight | Severe | Severe | Severe |
| | | | | | | | able | Hazy | Hazy | Hazy | Hazy | Hazy |
| | | | | | | | hazy | | | | | |
| % T | | | | 85.9 | 95.8 | 95.7 | 52.3 | 65.9 | 82 | 7.9 | 2.3 | 2.6 |

(1) Sodium Laureth sulfate, 27.5% active, Rhodapex® ES-STD, Rhodia, Cranbury, N.J., USA
(2) Cocamidopropyl betaine, 30% active, Amphosol® CA, Stepan Company, Northfield, Ill., USA
(3) DMDM Hydantoin, Glydant®, Lonza Inc. Fair Lawn, N.J., USA
(4) NaCl was added as 25% solution up to 2.5% active. Rest was added dry.
(5) ADPP5040 is an experimental cationic guar from Hercules Inc. It has Molecular weight of about 1,900,000 and cationic DS of about 1.3
(6) ADPP5199 is an experimental cationic guar from Hercules Inc. It has Molecular weight of about 1,600,000 and cationic DS of about 0.33.
(7) AQU D3798 is an experimental cationic guar from Hercules Inc. It has Molecular weight of about 460,000 and cationic DS of about 0.7

(8) GPX 247, is a commercial cationic guar from Hercules Inc. It has Molecular weight of 800,000

TABLE 8

| | X33712 | | | | | | | | | | | |
|---|---|---|---|---|---|---|---|---|---|---|---|---|
| | 37A | 37B | 37C | 37D | 37E | 37F | 38A | 38B | 38C | 38D | 38E | |
| | Wt | Wt | Wt | Wt | Wt | Wt | Wt | Wt | Wt | Wt | Wt | |
| Ingredients | (gr.) | (gr.) | (gr.) | (gr.) | (gr.) | (gr.) | (gr.) | (gr.) | (gr.) | (gr.) | (gr.) | 38F |
| Deionized | 25.31 | 25.31 | 25.31 | 25.31 | 25.31 | 25.31 | 25.31 | 25.31 | 25.31 | 25.31 | 25.31 | 25.31 |
| water | | | | | | | | | | | | |
| AQU | 0.50 | 0.50 | 0.50 | 0.50 | 0.50 | 0.50 | | | | | | |
| D3798 | | | | | | | | | | | | |
| GPX 247 | | | | | | | 0.50 | 0.50 | 0.50 | 0.50 | 0.50 | 0.50 |
| Sodium | 43.64 | 43.64 | 43.64 | 43.64 | 43.64 | 43.64 | 43.64 | 43.64 | 43.64 | 43.64 | 43.64 | 43.64 |
| Laureth | | | | | | | | | | | | |
| Sulfate | | | | | | | | | | | | |
| Cocamidopropyl | 10.00 | 10.00 | 10.00 | 10.00 | 10.00 | 10.00 | 10.00 | 10.00 | 10.00 | 10.00 | 10.00 | 10.00 |
| betaine | | | | | | | | | | | | |
| DMDM | 00.5 | 00.5 | 00.5 | 00.5 | 00.5 | 00.5 | 00.5 | 00.5 | 00.5 | 00.5 | 00.5 | 00.5 |
| Hydantoin | | | | | | | | | | | | |
| Sodium | 00.00 | 1.00 | 2.00 | 3.5 | 5.00 | 7.00 | 00.0 | 1.00 | 2.00 | 3.5 | 5.0 | 7.0 |
| Chloride | | | | | | | | | | | | |
| Deionized | 20.00 | 19.00 | 18.00 | 16.5 | 15.00 | 15.00 | 20.0 | 19.0 | 18.0 | 16.5 | 15.0 | 15.0 |
| Water | | | | | | | | | | | | |
| Total | 100 | 100 | 100 | 100 | 100 | 100 | 100 | 100 | 100 | 100 | 100 | 100 |
| batch | | | | | | | | | | | | |
| pH | | | 6.4 | 6.4 | 6.3 | 6.3 | | | | | | |
| Viscosity | | | 1300 | 14940 | 17660 | 42350 | | | | 13460 | 15580 | 42950 |
| (cps), | | | | | | | | | | | | |
| 30 rpm | | | | | | | | | | | | |
| Comment | Sep- | Sep- | | | | | Sep- | Sep- | Separated | | | |
| | arated | arated | | | | | arated | arated | | | | |
| % T | | | 75.5 | 86.4 | 30.5 | 3.4 | | | | 3.2 | 1.9 | 2.1 |

(1) Sodium Laureth sulfate, 27.5% active, Rhodapex® ES-STD, Rhodia, Cranbury, N.J., USA
(2) Cocamidopropyl betaine, 30% active, Amphosol® CA, Stepan Company, Northfield, Ill., USA
(3) DMDM Hydantoin, Glydant®, Lonza Inc. Fair Lawn, N.J., USA
(4) NaCl was added as 25% solution up to 2.5% active. Rest was added dry.
(5) ADPP5040 is an experimental cationic guar from Hercules Inc. It has Molecular weight of about 1,900,000 and cationic DS of about 1.3
(6) ADPP5199 is an experimental cationic guar from Hercules Inc. It has Molecular weight of about 1,600,000 and cationic DS of about 0.33.
(7) AQU D3798 is an experimental cationic guar from Hercules Inc. It has Molecular weight of about 466,000 and cationic DS of about 0.7
(8) GPX 247 is a commercial cationic guar from Hercules Inc. It has Molecular weight of 800,000 and cationic DS 0.11

### EXAMPLE 5

### Body Wash—Ammonium Laureth Sulfate/Cocamidopropyl betaine/Ammonium Chloride with ADPP 6486 and with N-Hance 3215 (Table 9)

Stock solution of body wash was prepared with 10% “hole” in it to add salt thickener at later stage as follows:
37.81 g Deionized water
0.50 g N-Hance® 3215
42.14 g Ammonium Laureth Sulfate (ALES)
9.00 g Cocamidopropyl betaine (CAPB)
00.50 g DMDM Hydantoin

N-Hance 3215 was added to water while mixing. Next, 5% citric acid solution was added to lower pH to about 6.0 and mixed for an hour. Next, ALES was added while mixing and was allowed to mix until homogeneous. Next, CAPB was added while mixing. Again, the mixture was allowed to mix until homogenous. Next, DMDM Hydantoin was added and mixed for about 10 minutes. The pH was adjusted with citric acid or sodium hydroxide solution to between 6 to 6.5.

To 90.0 grams of this body wash, 25% ammonium chloride solution was added to achieve the desired salt concentration in final body wash formulation. Water was added as necessary to adjust total weight to 100 grams. Dry salt was added when more than 2.5% of 100% active salt was added

Same procedure was followed for the polymer of this invention ADPP 6486

In Table 9, body wash formulated with cationic guar polymers of this invention ADPP6486 was compared in a body wash for their stability performance to commercially available cationic guar polymer N-Hance 3215 product. The body wash based on the commercial polymer, N-Hance 3215product, had poor stability as compared to the polymer of this invention over a broad range of salt concentration. The polymer of this invention showed a significant improvement in clarity and compatibility with increase in ammonium chloride salt. With the commercial product, the level of clarity using the product of the invention was unable to be achieved.

TABLE 9

| | X33768- | | | | | | | | | | | |
|---|---|---|---|---|---|---|---|---|---|---|---|---|
| | 17A | 17B | 17C | 17D | 17E | 17F | 18A | 18B | 18C | 18D | 18E | 18F |
| | Wt | Wt | Wt | Wt | Wt | Wt | Wt | Wt | Wt | Wt | Wt | Wt |
| Ingredients | (gr.) | (gr.) | (gr.) | (gr.) | (gr.) | (gr.) | (gr.) | (gr.) | (gr.) | (gr.) | (g) | (gr.) |
| Deionized | 37.81 | 37.81 | 37.81 | 37.81 | 37.81 | 37.81 | 37.81 | 37.81 | 37.81 | 37.81 | 37.81 | 37.81 |
| water | | | | | | | | | | | | |
| N- | 0.50 | 0.50 | 0.50 | 0.50 | 0.50 | 0.50 | | | | | | |
| Hance ® | | | | | | | | | | | | |
| 3215 | | | | | | | | | | | | |
| ADPP | | | | | | | 0.50 | 0.50 | 0.50 | 0.50 | 0.50 | 0.50 |
| 6486 | | | | | | | | | | | | |
| Ammonium | 42.14 | 42.14 | 42.14 | 42.14 | 42.14 | 42.14 | 42.14 | 42.14 | 42.14 | 42.14 | 42.14 | 42.14 |
| Laureth | | | | | | | | | | | | |
| Sulfate | | | | | | | | | | | | |
| Cocamidopropyl | 9.00 | 9.00 | 9.00 | 9.00 | 9.00 | 9.00 | 9.00 | 9.00 | 9.00 | 9.00 | 9.00 | 9.00 |
| betaine | | | | | | | | | | | | |
| DMDM | 00.5 | 00.5 | 00.5 | 00.5 | 00.5 | 00.5 | 00.5 | 00.5 | 00.5 | 00.5 | 00.5 | 00.5 |
| Hydantoin | | | | | | | | | | | | |
| Ammonium | 00.0 | 1.0 | 2.0 | 3.5 | 5.00 | 7.0 | 0.00 | 1.00 | 2.0 | 3.5 | 5.00 | 7.1 |
| Chloride | | | | | | | | | | | | |
| Deionized | 10.00 | 9.0 | 8.0 | 7.5 | 7.5 | 7.5 | 10.0 | 9.0 | 8.0 | 7.5 | 7.5 | 7.4 |
| Water | | | | | | | | | | | | |
| Total | 100 | 100 | 100 | 100 | 100 | 100 | 100 | 100 | 100 | 100 | 100 | 100 |
| batch | | | | | | | | | | | | |
| pH | 6.2 | 6.2 | 6.4 | 6.5 | 6.4 | 6.3 | 6.1 | 6.1 | 6.2 | 6.3 | 6.2 | 6.2 |
| Viscosity | 490 | 1344 | 4780 | 13820 | 18240 | 13020 | 86 | 98 | 1860 | 10780 | 15200 | 11280 |
| (cps), | | | | | | | | | | | | |
| 30 rpm | | | | | | | | | | | | |
| Comment | Mod- | Mod- | Mod- | Considerable | Sever | Separation | Sep- | Sep- | Sever | Slight | Clear | Clear |
| | erate | erate | erate | Haze | Haze. | | arated | arated | haze | Haze | | |
| | Haze | Haze | Haze | | [symbol] | | | | | | | |
| % T | 42 | 49 | 46 | 7 | 7 | — | — | — | 1.5 | 79 | 96 | 96 |

(1) ADPP6486 is an experimental material with 1,920,000 molecular weight and cationic DS of 2.1 from Hercules Inc, Wilmington, Del.
(2) N-Hance® 3215 from Hercules Inc. Wilmington, Del.
(3) Aluminum Laureth sulfate, 28% active, Steol® CA330, Stepan Company, Northfield, Ill., USA
(4) Cocamidopropyl betaine, 30% active, Amphosol® CA, Stepan Company, Northfield, Ill., USA
(5) DMDM Hydantoin, Glydant®, Lonza Inc. Fair Lawn, N.J., USA

### EXAMPLE 6

### Body Wash—Sodium Laureth Sulfate/Cocamidopropyl betaine/Sodium Chloride with N-Hance® 3215 and with ADPP 6486 (Table 10)

Stock body wash solution was made with 10% “hole” in it to add salt thickener at later stage as follows:
35.31 g Deionized water
0.50 g N-Hance® 3215
43.64 g Sodium Laureth Sulfate (SLES)
10.00 g Cocamidopropyl betaine (CAPB)
00.50 g DMDM Hydantoin

To deionized water, N-Hance® commercial 3215 product was added while mixing. Next, 5% citric acid solution was added to lower pH to about 6.0 and mixed for an hour or until polymer dissolved. Next, SLES was added slowly while mixing and allowed to mix until homogeneous. Next, CAPB was added while mixing. Again, the mixture was allowed to mix until homogenous. Next, DMDM Hydantoin was added and mixed for about 10 minutes. The pH was adjusted with citric acid or sodium hydroxide solution to between 9 to 9.6.

To 90.0 grams of this body wash solution, 10.0 grams of 25% sodium chloride solution was added to achieve the desired salt concentration in final body wash formulation. Additional salt was added as dry salt when necessary to achieve desired salt concentration

The body wash viscosity was measured with Brookfield® LVT viscometer. Product stability was visually observed for homogeneity, insoluble gels or phase separation. Body wash clarity was visually rated as being clear, very slightly hazy slightly hazy, moderate hazy, hazy, very hazy, considerable hazy, sever hazy, and opaque. This type of rating is common in the Personal Care Industry for comparative studies. The clarity was also measured at 600 nm using a Spectrophotometer, Cary 5E UV-VIS-NIR, available from Varian Instruments, Inc., or equivalent. The clarity measurements at 600 nm wavelength are reported as % T value. The higher the number, the clearer is the solution.

Addition of salt such as sodium chloride improved the clarity and stability of the body wash containing the guar product of this invention (i.e., ADPP 64-86). This was not the case with commercially available N-Hance 3215 product. In fact, the body wash became unstable with the addition of salt.

| | X33768- | | | | | | | |
|---|---|---|---|---|---|---|---|---|
| | 26A | 26B | 26C | 26D | 27A | 27B | 27C | 27D |
| | Wt | Wt | Wt | Wt | Wt | Wt | Wt | Wt |
| Ingredients | (gr.) | (gr.) | (gr.) | (gr.) | (gr.) | (gr.) | (gr.) | (gr.) |
| Deionized | 35.31 | 35.31 | 35.31 | 35.31 | 35.31 | 35.31 | 35.31 | 35.31 |
| water | | | | | | | | |
| N- | 0.50 | 0.50 | 0.50 | 0.50 | | | | |
| Hance ® | | | | | | | | |
| 3215 | | | | | | | | |
| ADPP | | | | | 0.5 | 0.5 | 0.5 | 0.5 |
| 6486 | | | | | | | | |
| Sodium | 43.63 | 43.63 | 43.63 | 43.63 | 43.63 | 43.63 | 43.63 | 43.63 |
| Laureth | | | | | | | | |
| Sulfate | | | | | | | | |
| Cocamidopropyl | 10.00 | 10.00 | 10.00 | 10.00 | 10.00 | 10.00 | 10.00 | 10.00 |
| betaine | | | | | | | | |
| DMDM | 00.5 | 00.5 | 00.5 | 00.5 | 00.5 | 00.5 | 00.5 | 00.5 |
| Hydantoin | | | | | | | | |
| Sodium | 00.0 | 1.0 | 3.5 | 7.0 | 00.0 | 1.0 | 3.5 | 7.0 |
| Chloride | | | | | | | | |
| Deionized | 10.00 | 9.0 | 7.5 | 7.5 | 10.00 | 9.0 | 7.5 | 7.5 |
| Water | | | | | | | | |
| Total | 100 | 100 | 100 | 105 | 100 | 100 | 100 | 100 |
| batch | | | | | | | | |
| pH | 9.3 | 9.5 | 9.1 | 8.9 | 9.3 | 9.3 | 9 | 8.9 |
| Viscosity | 421 | 1028 | 180000 | 333200 | | | 15620 | 82100 |
| (cps), | | | | | | | | |
| 30 rpm | | | | | | | | |
| Comment | Hazy | Hazy | Separated | Gelled | Separated | Separated | Hazy | Slightly |
| | | | | | | | | Hazy |
| % T | 46 | 50 | | | | | 83 | 87 |

Ingredients
(1) Cationic guar, 100% active, N-Hance® 3215, Aqualon, Wilmington, Del., USA. It has weight average molecular weight of about 1,300,000 and cationic Ds of about 0.21
(2) ADPP 6486, lot 33360-89-2 has cationic DS of 2.1 and the weight average Molecular weight of 1,920,000.
(2) Sodium Laureth sulfate, 27.5% active, Rhodapex® ES-STD, Rhodia, Cranbury, N.J., USA
(3) Cocamidopropyl betaine, 30% active, Amphosol® CA, Stepan Company, Northfield, Ill., USA
(4) DMDM Hydantoin, Glydant®, Lonza Inc. Fair Lawn, N.J., USA
(5) NaCl was added as 25% solution up to 2.5% active. Rest was added dry.

### EXAMPLE 7

### Preparation of Body Wash with N-Hance® 3215 and with ADPP6486, Lot# X33360-89-2 (Table 11)

N-Hance® 3215 and ADPP6486 polymers were added to water while mixing. Next pH was lowered to about 6 with citric acid solution. Mixing was continued until polymer dissolved. Next, ammonium lauryl sulfate, ammonium laureth sulfate, cocamide MEA, Methyl Gluceth-20 and PEG-120 Methyl glucose dioleate, DMDM Hydantoin were added in the order listed. Next body wash pH was adjusted to about 6 with NaOH solution.

The addition of commercial N-Hance® 3215 increased viscosity of body was significantly compared to the body wash made with the polymer of this invention, ADPP6486. That is for a body wash formulation where conditioning cationic guar is desired, commercial N-Hance® 3215 would be undesirable due to significant increase in viscosity. A very high viscosity can make a difficult to dispense the product. In fact, polymer of this invention had no significant effect on the body wash viscosity when compared to body wash without the polymer of invention. The additions of polymer of this invention improve foam stability of the body wash over the body wash without any conditioning polymer. The lather drainage time for body wash without the polymer of this invention was 31 seconds (30, 33 and 30 seconds) as compared to 42 seconds (48, 41, 45 30 and 44 seconds) with polymer of this invention. A longer the lather drainage time, the better is the lather stability.

TABLE 11

| | X33768- | | |
|---|---|---|---|
| | 34A | 34B | 34C |
| | Wt | Wt | Wt |
| Ingredients | (gr.) | (gr.) | (gr.) |
| Deionized water | 24.78 | 24.78 | 24.78 |
| N-Hance ® 3215 | 0.00 | 0.20 | |
| ADPP6486 | | | 0.20 |
| Ammonium lauryl sulfate | 13.93 | 13.93 | 13.93 |
| Ammonium laureth sulfate | 51.85 | 51.85 | 51.85 |
| Cocamidopropyl betaine | 4.0 | 4.0 | 4.0 |
| Cocamide MEA | 2.5 | 2.5 | 2.5 |
| Methyl Gluceth 20 | 0.5 | 0.5 | 0.5 |
| PEG-120 methyl Glucose | 1.5 | 1.5 | 1.5 |
| Dioleate | | | |
| DMDM Hydantoin | 00.5 | 00.5 | 00.5 |
| Total batch | 100 | 100 | 100 |
| pH | 6 | 6 | 6 |
| Viscosity (cps), 30 rpm | 79900 | 236800 | 68900 |
| Comment | Slt haze | Moderate Haze | Sever Haze |
| % T | 92 | 83.3 | |

Ingredients
(1) Cationic guar, 100% active, N-Hance® 3215, Aqualon, Wilmington, Del., USA. It has weight average molecular weight of about 1,300,000 and cationic Ds of about 0.21.
(2) ADPP6486, lot 33360-89-2 has cationic DS of 2.1 and the weight average Molecular weight of 1,920,000.
(3) Ammonium Lauryl sulfate—Stepanol® AM, Stepan Company Northfield, Ill. 60093
(4) Ammonium laureth Sulfate—Steol® CA330, Stepan Company Northfield, Ill. 60093
(5) Cocamidopropyl betaine—Amphosol® CA, Stepan Company Northfield, Ill. 60093
(6) Cocamide MEA—Monamid® CMA, Uniqema, Newcastle, Del.
(7) Methyl Gluceth-20—Glucam® E-20, Dow Chemicals, Midland Mich.
(8) PEG-120 Methyl Glucose Dioleate—Glucamate® DOE-120, Dow Chemicals, and Midland Mich.
(9) DMDM Hydantoin, Glydant®, Lonza Inc. Fair Lawn, N.J., USA
(10) NaCl was added as 25% solution up to 2.5% active. Rest was added dry.
   Lather Drainage Test:

Objective of this is to measure the lather drainage time of a diluted body wash solution. Long drainage times indicate a rich, dense lather with good stability. The test was used to determine the influence polymers of this invention may have on lather quality.

Equipment:

Waring® Blender Model #7012 or 34BL97 or equivalent.
Funnel, preferably plastic; 6″ diameter, ⅞″ ID neck, 5¼″ high, with a horizontal wire 2″ from the top.
U.S.A. Standard Testing Sieve NO.20 or Tyler® Equivalent 20 mesh or 850 micrometer or 0.0331 inch sieve. Preferably over 7 inch in diameter but smaller size could also be used Stopwatch or a timer.
   Procedure:

For each test formulation, 100 g of a diluted body wash solution was prepared as shown below.
Body wash 066.13 g
Deionized Water 933.87 g
Total 1000.00 g

1. For each lather test measurement 200 grams of above diluted solution was weighed and placed in a 25° C. water-bath for 2 hours. Total of three jars each with 200 grams of solutions were prepared per body wash formulation

2. Next, the lather drainage time for each solution was measured using the procedure described below.
a. Poured 200 g of solution into a clean, dry Waring blender glass vessel.
b. Whipped at the highest speed for exactly 1 minute while covered.
c. Foam generated in the jar was immediately poured into a clean, dry funnel standing on a 20 mesh screen over a beaker.
d. Foam from the blender was poured for exactly 15 seconds. The goal is to get as much foam as possible into the funnel without overflowing. After 15 seconds stopped pouring foam however, the stopwatch was kept running.
e. The total time needed for the foam to drain including the 15 seconds for pour time was recorded once the wire was no longer covered by foam or liquid.

### EXAMPLE 8

### Preparation of Body Wash with N-Hance® 3215 and with ADPP6486, (Table 12)

N-Hance® 3215 and ADPP6486 polymers were added to water while mixing. Next pH was lowered to about 6 with citric acid solution. Mixing was continued until polymer dissolved. Next, Tetra sodium EDTA, sodium chloride, C9-C15 alkyl phosphate, sodium laureth sulfate, sodium lauryl sulfate and PPG-2 hydroxyethyl Cocamide, DMDM Hydantoin were added in the order listed. Next body wash pH was adjusted to about 6 with NaOH solution.

The addition of commercial N-Hance® 3215 increase viscosity of body was significantly compared to body wash made with the polymer of this invention, ADPP6486. That is for a body wash formulation were conditioning cationic guar is desired, commercial N-Hance® 3215 would be undesirable due to it negative effect on the body wash viscosity. The additions of polymer of this invention improve foam stability of the body wash over the body wash without any conditioning polymer. The lather drainage time for the body wash without the polymer was 58 Seconds (48, 48, 64, 55, 74 seconds) compared to 80 Seconds (88, 70, and 64, 76 and 103 seconds) with the polymer of this invention.

TABLE 12

| | X33768- | | |
|---|---|---|---|
| | 36A | 36B | 36C |
| | Wt | Wt | Wt |
| Ingredients | (gr.) | (gr.) | (gr.) |
| Deionized water | 24.78 | 24.78 | 24.78 |
| N-Hance ® 3215 | 0.00 | 0.2 | |
| ADPP6486 | 0.00 | | 0.20 |
| Tetra sodium EDTA | 0.3 | 0.3 | 0.3 |
| Sodium Chloride | 1.0 | 1.0 | 1.0 |
| C9-C15 Alkyl phosphate Sodium | 6.7 | 6.7 | 6.7 |
| laureth Sulfate | | | |
| Sodium laureth Sulfate | 46.4 | 46.4 | 46.4 |
| Sodium Lauryl sulfate | 27.00 | 27.00 | 27.00 |
| PPG-2 Hydroxyethyl Cocamide | | | |
| DMDM Hydantoin | 00.5 | 00.5 | 00.5 |
| Total batch | 100 | 100 | 100 |
| pH | 6 | 6 | 6 |
| Viscosity (cps), 30 rpm | 14220 | 44650 | 13100 |
| Comment | Slt haze | Moderate | Sever |
| | | Haze | Haze |
| % T | 99 | 85 | 1.6 |

Ingredients
(1) Cationic guar, 100% active, N-Hance® 3215, Aqualon, Wilmington, Del., USA. Molecular weight 1,300,000 and cationic DS 0.21.
(2) ADPP 6486 has cationic DS of 2.1 and the weight average Molecular weight of 1,920,000.
(3) Sodium Lauryl sulfate—Stepanol® WAC, Stepan Company Northfield, Ill. 60093
(4) Sodium laureth Sulfate—Rhodapex® ES-2, Rhodia, Cranbury, N.J. 08512
(5) Cocamidopropyl betaine—Amphosol® CA, Stepan Company Northfield, Ill. 60093
(6) PPG-2 Hydroxyethyl Cocamide—Promidium® CO, Uniqema, Newcastle, Del.
(7) Tetra Sodium EDTA—Fisher Scientific
(9) DMDM Hydantoin, Glydant®, Lonza Inc. Fair Lawn, N.J., USA
(10) Sodium Chloride from Baker.

### EXAMPLE 9

### Skin Moisturizinq Body Wash with N-Hance® 3215 and with ADPP6486 (Table 13)

Disodium EDTA was first dissolved in water. Next Carbomer, propylene glycol, glycerin, sodium laureth sulfate, Disodium dimethicone copolyol sulfosuccinate, and poysorbate 20, blend of dimethicone, laureth-4, laureth-23, Cocamidopropyl betaine, cationic guar, and DMDM Hydantoin were added in the order listed. Between each addition, sufficient time was allowed for homogenous mixing. Next pH was adjusted to about 6.5 with Triethanolamine

The body washes without the polymer and with the polymer of this invention were smooth, homogenous appearance. However, body wash made with the commercial cationic guar N-Hance® 3215 was nonhomogenious and lumpy. That is the commercial cationic was not compatible.

TABLE 13

| | X33768- | | |
|---|---|---|---|
| | 37A | 37B | 37C |
| | Wt | Wt | Wt |
| Ingredients | (gr.) | (gr.) | (gr.) |
| Deionized water | 54.4 | 54.4 | 54.4 |
| Disodium EDTA | 0.05 | 0.05 | 0.05 |
| Acrylates/C10-C30 Alkyl acrylate cross | 0.5 | 0.5 | 0.5 |
| polymer | | | |
| Propylene glycol | 1.0 | 1.0 | 1.0 |
| Glycerine | 2.0 | 2.0 | 2.0 |
| Sodium laureth sulfate | 16.0 | 16.0 | 16.0 |
| Disodium dimethicone copolyol | 15.0 | 15.0 | 15.0 |
| sulfosuccinate | | | |
| Poysorbate 20 | 1.0 | 1.0 | 1.0 |
| Dimethicone & Laureth-4 & Laureth-23 | 5.0 | 5.0 | 5.0 |
| Cocamidopropyl betaine | 3.0 | 3.0 | 3.0 |
| N-Hance 3215 | 0.0 | 0.20 | 0.0 |
| ADPP6486 | 0.0 | 0.0 | 0.20 |
| Triethanolamine amine | 1.1 | 1.1 | 1.1 |
| DMDM Hydantoin | 0.5 | 0.5 | 0.5 |
| Total | 100 | 100 | 100 |
| Viscosity (cps) | 3564 | 5260 | 4480 |
| Comment | White, | White | White |
| | smooth, | lumpy, | smooth, |
| | creamy, | non | creamy, |
| | Opaque | homogen | Opaque |
| pH | 6.6 | 6.7 | 6.6 |

Ingredients
(1) Cationic guar, 100% active, N-Hance® 3215, Aqualon, Wilmington, Del., USA. Molecular weight 1,300,000 and cationic DS 0.21
(2) ADPP 6486, lot 33360-89-2 has cationic DS of 2.1 and the weight average Molecular weight of 1,920,000.
(3) Acrylates/C10-C30 Alkyl acrylate cross polymer—Carbopol® ETD 2020, Noveon Cleveland, Ohio
(4) Sodium laureth Sulfate—Rhodapex® ES-2, Rhodia, Cranbury, N.J. 08512
(5) Cocamidopropyl betaine—Amphosol® CA, Stepan Company Northfield, Ill. 60093
(6) Poysorbate 20—Tween® 20, Uniqema, Newcastle, Del.
(7) Disodium dimethicone copolyol sulfosuccinate—Mackanate® DC-30, McIntyre Group, University Park, Ill.
(8) Disodium EDTA—Hampene Na2, Hampshire Chemical Corp, Subsidiary of Dow Chemicals, Nashua, N.H.
(9) Dimethicone & Laureth-4 & Laureth-23—SM2169 GE Silicones
(10) Triethanolamine amine—Baker
(11) DMDM Hydantoin, Glydant®, Lonza Inc. Fair Lawn, N.J., USA

### EXAMPLE 10

### Shower Gel with Jaguar® Excel and with AQU D3798 Lot# X32981-76A (Table 14)

In container 1, PEG-150 pentaerythrityl tetra acetate was added to water and then heated to 75° C. and mixed until homogeneous. Next propylene glycol was added. In container 2, SLES, ALES, CAPB, Lauramide DEA, silicone elastomer were mixed together and mixed until homogenous. Next mixture of container two was added to container 1 while mixing. And then DMDM Hydantoin was added. To the mixture Jaguar® Excel or polymer of this invention AQU D3798 lot# X32981-76A was added. Next citric acid was added to adjust pH between 6 to 6.5. Temperature of the solution was lowered to room temperature while stirring.

The polymer of this invention had little effect on the viscosity of this formulation as compared to Jaguar® Excel polymer. The polymer of this invention can be added at a higher level if desired for heavier conditioning as compared to Jaguar Excel polymer.

TABLE 14

| | X33768- | | |
|---|---|---|---|
| | 46A | 46B | 46C |
| | Wt | Wt | Wt |
| Ingredients | (gr.) | (gr.) | (gr.) |
| Deionized water | 47.11 | 47.11 | 47.11 |
| PEG-150 pentaerythrityl tetra | 1.5 | 1.5 | 1.5 |
| acetate | | | |
| Propylene glycol | 1.0 | 1.0 | 1.0 |
| Sodium laureth sulfate (SLES) | 20.0 | 20.0 | 20.0 |
| Ammonium laureth | 15.0 | 15.0 | 15.0 |
| sulfate(ALES) | | | |
| Cocamidopropyl betaine (CAPB) | 8.0 | 8.0 | 8.0 |
| Lauramide DEA | 4.0 | 4.0 | 4.0 |
| Silicone elastomer blend | 2.0 | 2.0 | 2.0 |
| DMDM Hydantoin | 0.5 | 0.5 | 0.5 |
| Jaguar Excel | 0.0 | 0.4 | 0.0 |
| AQU D3798 | 0.0 | 0.0 | 0.4 |
| Total | 100 | 100 | 100 |
| pH | 6.3 | 6.3 | 6.1 |
| Viscosity (cps) | 2464 | 5140 | 2780 |
| Comment | Moderate | Sever Haze | Sever |
| | Haze | | Haze |

Ingredients
(1) Cationic guar—100% active, Jaguar® Excel, Rhodia, Cranbury, N.J. 08512
(2) AQU D3798 Cationic DS 0.70 and the weight average Molecular weight of 460,000. Hercules Inc, Wilmington, Del.
(3) Sodium laureth sulfate—Rhodapex® ES-2 Rhodia, Cranbury, N.J. 08512
(4) Ammonium laureth sulfate—Steol® CA330, Stepan Company Northfield, Ill. 60093
(5) Cocamidopropyl betaine—Amphosol® CA, Stepan Company Northfield, Ill. 60093
(6) Lauramide DEA—Standamid® LD, Amerchol, Div of Dow Chemicals, Piscataway, N.J.
(7) Cyclopentasiloxane & PEG-12 dimethicone cross polymer—Silicone elastomer blend—Dow Corning Corp. Midland, Mich.
(8) DMDM Hydantoin, Glydant®, Lonza Inc. Fair Lawn, N.J., USA

### EXAMPLE 11

### Body Wash with Jaguar® C162 and ADPP6486 (Table 15)

In container 1, Jaguar C162 polymer and polymer of this invention ADPP6486 were added to water and pH was lowered to 6.0 to 6.5. The solution was mixed until the polymer fully dissolved. In container 2, SLES, Disodium EDTA, Cetearyl alcohol and tricetylmethyl chloride were mixed together. Mixture of container 2 was added to container 1 while stirring and heated to 80° C. In a third container, 0.3 grams of Xanthan gum was added to 9.7 grams of water and mixed for 40 minutes. Next, CAPB, Glycol distearate, Laureth-4, cocamidopropyl betaine, formic acid Dimethicone, laureth-23 and laureth-3 and sodium laureth sulfate are added and mixed for one hour. While the ingredients in the third container was mixing, the mixture in container 1 was cooled while still under agitation by turning off the heat. Next, mixture of container 3 was added to container 1 and mixed for 15 minutes. Body wash pH was adjusted to between 6 to 6.5. Sodium chloride was added optionally for stability and viscosity.

The polymer of this invention, ADPP6486, had very little effect on final product viscosity as compared to the commercial cationic guar Jaguar® C162 product.

TABLE 15

| | 48A | 48B | 48C |
|---|---|---|---|
| | Wt | Wt | Wt |
| Ingredients | (gr.) | (gr.) | (gr.) |
| Deionized water | 47.11 | 47.11 | 47.11 |
| Jaguar C162 | 0.0 | 0.3 | 0.0 |
| ADPP6486 | 0.0 | 0.0 | 0.30 |
| Sodium Laureth Sulfate (SLES) | 24.0 | 24.0 | 24.0 |
| Disodium EDTA | 0.1 | 0.1 | 0.1 |
| Cetearyl alcohol | 0.6 | 0.6 | 0.6 |
| Tricetylmethyl Chloride | 0.2 | 0.2 | 0.2 |
| Cocamidopropyl betaine (CAPB) | 6.0 | 6.0 | 6.0 |
| Xanthan gum (3% solution) | 10.0 | 10.0 | 10.0 |
| Glycol distearate, Laureth-4, | 5.0 | 5.0 | 5.0 |
| Cocamidopropyl betaine, formic acid | | | |
| Dimethicone and laureth-23 and laureth-3 | 4.0 | 4.0 | 4.0 |
| and sodium laureth sulfate | | | |
| DMDM Hydantoin | 0.5 | 0.5 | 0.5 |
| Total | 100 | 100 | 100 |
| pH | 6.3 | 6.3 | 6.2 |
| Viscosity (cps) | 320 | 630 | 390 |
| Comment | Opaque | Opaque | Opaque |

Ingredients
(1) Cationic guar—100% active, Jaguar® C162, Rhodia, Cranbury, N.J. 08512. Molecular weight 800,000 and cationic DS 0.1
(2) Cationic guarADPP6486, Molecular weight 1,920,000, Cationic DS 2.1. Hercules Inc, Wilmington, Del.
(3) Sodium laureth sulfate—Rhodapex® ES-STD Rhodia, Cranbury, N.J. 08512
(4) Disodium EDTA—Hampene® Na2, Hampshire Chemical Corp, Subsidiary of Dow Chemicals, Nashua, N.H.
(5) Cetearyl alcohol—Lanette® O, Cognis Corp., amber, PA
(6) Tricetylmethyl Chloride—Arquad 316, Akzo-Nobel coatings, Inc. Somerset, N.J.
(7) Cocamidopropyl betaine—Amphosol CA, Stepan Company Northfield, Ill. 60093
(8) Lauramide DEA—Standamid LD, Amerchol, Div of Dow Chemicals, Piscataway, N.J.
(9) Xanthan gum—Kelco K6B166—CP Kelco, Wilmington, Del.
(10) Glycol distearate, Laureth-4, Cocamidopropyl betaine, formic acid—Euperlan PK3000AM, Cognis Corp., amber, PA
(11) Dimethicone and laureth-23 and laureth-3 and sodium laureth sulfate—Dow Corning 2-1352, Dow Corning Corp. Midland, Mich.
(12) DMDM Hydantoin, Glydant®, Lonza Inc. Fair Lawn, N.J., USA

### EXAMPLE 12

### After Shower Skin Conditioning Liquid (Table 16)

In container 1, hydroxyethyl cellulose was added to water and the pH was adjusted to about 8.5. The mixture was stirred for 30 minutes or until the mixture became a clear solution. Next, conditioning cationic polymer AQU D3799 and AQU D3939 were added and mixed for an additional 30 minutes. This mixture was heated to 65° C. until the mixture became homogenous. Next, cetyl alcohol was added and mixed until mixture became homogeneous and then it was cooled to 50° C. while mixing. Potassium chloride was next added. Isopropyl myristate was then added and mixed until the mixture became homogeneous. The pH was then adjusted to between 5.25 and 5.5. Afterwards, preservative was added and cooled to room temperature while mixing.

To determine a conditioning properties of a polymer, hairs are often treated with a conditioning agent dissolved or suspended in water and then hairs combed to measure force required to comb hair. A lower the combing force better the conditioning properties. Some time hair treated with fully formulated product containing conditioning agent are also used by both product manufacturers and the formulators. A lower the combing force, better the conditioning properties of a polymer. The following conditioning formula without the polymer required high stress to comb wet hair, about 4500 gf-mm/g of hair compared to only about 600 to 900 gf-mm/g of hair for the formulation with polymer of this invention. Similar trend was also observed for the dry hair. The following conditioning formula without the polymer required high stress to comb dry hair, about 325 gf-mm/g of hair compared to only about 140 gf-mm/g of hair for the formulation with polymer of this invention. This is a strong indication that polymer provides excellent conditioning. A commercial polymer GPX 247 provided combing energy for wet hair about 1390 gf-mm/g and for dry hair about 260 gf-mm/g of hair

TABLE 16

| | 16-1 | 17-3 | 69-1 | 69-2 |
|---|---|---|---|---|
| | Wt | Wt | Wt | Wt |
| Ingredients | (gr.) | (gr.) | (gr.) | (gr.) |
| Phase 1 | | | | |
| Deionized Water | 188.2 | 188.2 | 188.2 | 188.2 |
| Hydroxyethyl cellulose | 1.4 | 1.4 | 1.4 | 1.4 |
| GPX 247 | 0.0 | 0.4 | 0.0 | 0.0 |
| AQU D3799 cationic guar | 0.0 | 0.0 | 0.4 | 0.0 |
| AQU D3939 cationic guar | 0.0 | 0.0 | 0.0 | 0.4 |
| Cetyl alcohol | 4.0 | 4.0 | 4.0 | 4.0 |
| Potassium chloride | 1.0 | 1.0 | 1.0 | 1.0 |
| Isopropyl myristate | 4.0 | 4.0 | 4.0 | 4.0 |
| Citric acid, 5%, as needed to adjust | | | | |
| pH | | | | |
| NaOH, 5%, as needed to adjust pH | | | | |
| Preservative | 1.0 | 1.0 | 1.0 | 1.0 |
| Total adjust with water | 200 | 200 | 200 | 200 |
| pH | 5.4 | 5.4 | 5.2 | 5.2 |
| Viscosity | 990 | 1330 | 1930 | 1700 |

Ingredients
(1) AQU D3799 has cationic DS of 0.63 and the weight average Molecular weight of 1,120,000. Hercules Inc. Wilmington, De
(2) AQU D3939 has cationic DS of 1.01 and the weight average Molecular weight of 1,750,000. Hercules Inc, Wilmington, Del.
(3) GPX 247 has cationic DS of 0.13 and the weight average Molecular weight of 500,000 from Hercules Inc, Wilmington, Del.
(4) Hydroxyethyl cellulose—Natrosol® 250HHR, Hercules Inc. Wilmington, Del.
(5) Cetyl Alcohol—Crodacol® C95NF, Croda, Inc Parsippany, N.J.
(6) Potassium Chloride—Fischer Scientific
(7) Isopropyl myristate—Stepan® IPM, Stepan Co, Northfield, Ill.
(8) Preservative—Germaben® II—ISP Wayne, N.J.

### EXAMPLE 13

Hand and Body Lotion with Commercial Cationic guar

| | | Weight | |
|---|---|---|---|
| | | % | |
| Ingredient | | Active | Function |
| A. | Natrosol ® Plus CS, Grade 330 | 0.50 | Rheology, Co- |
| | | | emulsifier |
| | Cationic guar TS = 10% | 0.25 | conditioner |
| | Distilled water | 78.00 | solvent |
| | Glycerin, | 2.00 | humectant |
| B. | Glycol stearate (Alkamuls ® EGMS) | 2.75 | emulsifier, opacifier |
| | Stearic acid (Industrene ® 5016) | 2.50 | surfactant, |
| | | | emulsifier |
| | Mineral oil (Drakeol ® 7) | 2.00 | emollient |
| | Acetylated lanolin (Lipolan ® 98) | 0.50 | skin/hair conditioner |
| | Cetyl alcohol (Crodacol ® C95) | 0.25 | surf., emulsifier, |
| | | | opacifier |
| C. | Distilled water | 10.00 | solvent |
| | Triethanolamine | 0.50 | pH adjust |
| D. | Propylene glycol and diazolidinyl | 0.75 | biocide |
| | urea and methylparaben and | | |
| | propylparaben | | |
| | | 100.00 | |

Procedure:

1. Natrosol® Plus polymer was dispersed in water by adding to the vortex of well-agitated from Part A. It was mixed for five minutes. Next solution pH was raised between 8 to 8.5 with NaOH and mixed until Natrosol Plus was fully dissolved. Next cationic guar was added and pH was lowered to about 7 to 7.5. Mixed the solution until cationic guar is dissolved. Next, glycerin was added with continued mixing and heated to 80° C. Mixed 15 minutes at 80° C.

2. In a separate vessel, blended Part B ingredients. And heated to 80° C. and mixed well.

3. Added Part A to Part B with good agitation while maintaining emulsion temperature at 80° C.

4. Combined Part C ingredients. And added to emulsion. Mixed continuously while cooling to 40° C.

5 Adjusted pH between 6.0 to 6.5

6. Added Part D (preservative) to emulsion. Mixed well.

7. Cooled and filled.

Ingredient List:

Commercial cationic guar, Molecular weight 50,000; cationic DS 0.12 from Hercules Inc
Alkamuls® EGMS: Rhodia Corp
Inustrene® 5016: Witco Corporation
Drakeol® 7: Penreco, Pennzoil Products Company
Lipolan 98: Lipo Chemicals
Crodacol® C95: Croda Inc
Natrosol® Plus—Hercules Inc.
Comment on Appearance: Glossy, smooth and creamy pH=6.31
RVT/Helipath @ 5 RPM, Spindle #94=113,600 cP.

### EXAMPLE 14

Hand and Body Lotion with and Cationic guar of this invention

| Ingredient | | Weight % | | Function |
|---|---|---|---|---|
| A. | Natrosol Plus CS, Grade 330 | 0.50 | | Rheology, Co- |
| | | | | emulsifier |
| | Cationic guar (X32838-60A, | 0.25 | | conditioner |
| | TS = 5%) | | | |
| | Distilled water | 78.00 | | solvent |
| | Glycerin, | 2.00 | | humectant |
| B. | Glycol stearate (Alkamuls EGMS) | 2.75 | | emulsifier, |
| | | | | opacifier |
| | Stearic acid (Industrene 5016) | 2.50 | | surfactant, |
| | | | | emulsifier |
| | Mineral oil (Drakeol 7) #K3103 | 2.00 | | emollient |
| | Aetylated lanolin (Lipolan 98) | 0.50 | | skin/hair |
| | | | | conditioner |
| | Cetyl alcohol (Crodacol C95) | 0.25 | | surf., |
| | | | | emulsifier, opacifier |
| C. | Distilled water | 10.00 | | solvent |
| | Triethanolamine | 0.50 | | pH adjust |
| D. | Propylene glycol and diazolidinyl | 0.75 | | biocide |
| | urea and methylparaben and | | | |
| | propylparaben | | | |
| | Total | 100.0 | g | |

Procedure:
1. Natrosol® Plus polymer was dispersed in water by adding to the vortex of well-agitated from Part A. It was mixed for five minutes. Next solution pH was raised between 8 to 8.5 with NaOH and mixed until Natrosol Plus was fully dissolved. Next cationic guar was added and pH was lowered to about 7 to 7.5. Mixed the solution until cationic guar is dissolved. Next, glycerin was added with continued mixing and heated to 80° C. Mixed 15 minutes at 80° C.
2. In a separate vessel, blended Part B ingredients. And heated to 80° C. and mixed well.
3. Added Part A to Part B with good agitation while maintaining emulsion temperature at 80° C.
4. Combined Part C ingredients. And added to emulsion. Mixed continuously while cooling to 40° C.
5 Adjusted pH between 6.0 to 6.5
6. Added Part D (preservative) to emulsion. Mixed well.
7. Cooled and filled.
   Ingredient List:
Cationic guar of this invention, Molecular weight 62,000, Cationic Ds 0.49 from Hercules Incorporated
Alkamuls® EGMS: Rhodia Corp
Inustrene® 5016: Witco Corporation
Drakeol® 7: Penreco, Pennzoil Products Company
Lipolan 98: Lipo Chemicals
Crodacol® C95: Croda Inc
Natrosol® Plus—Hercules Inc.
Comment on Appearance: Glossy with slightly grainy texture and appearance. pH=6.04 RVT/Helipath @ 5 RPM, Spindle #94=120,400 cP.

### EXAMPLE 15

Hand and Body Lotion with commercial Cationic guar

| Ingredient | | Weight % | Function |
|---|---|---|---|
| A. | Natrosol Plus CS, Grade 330 | 0.50 | Rheology, Co- |
| | | | emulsifier 5 |
| | Cationic guar (NH 3215) | 0.25 | conditioner |
| | Distilled water | 78.00 | solvent |
| | Glycerin, | 2.00 | humectant |
| B. | Glycol stearate (Alkamuls EGMS) | 2.75 | emulsifier, opacifier |
| | Stearic acid (Industrene 5016) | 2.50 | surfactant, |
| | | | emulsifier |
| | Mineral oil (Drakeol 7) | 2.00 | emollient |
| | Acetylated lanolin (Lipolan 98) | 0.50 | skin/hair conditioner |
| | Cetyl alcohol (Crodacol C95) | 0.25 | surf., emulsifier, |
| | | | opacifier |
| C. | Distilled water | 10.00 | solvent |
| | Triethanolamine | 0.50 | pH adjust |
| D. | Propylene glycol and diazolidinyl | 0.75 | biocide |
| | urea and Methylparaben and | | |
| | propylparaben | | |
| | | 100 | |

Procedure:
1. Natrosol® Plus polymer was dispersed in water by adding to the vortex of well-agitated from Part A. It was mixed for five minutes. Next solution pH was raised between 8 to 8.5 with NaOH and mixed until Natrosol Plus was fully dissolved. Next cationic guar was added and pH was lowered to about 7 to 7.5. Mixed the solution until cationic guar is dissolved. Next, glycerin was added with continued mixing and heated to 80° C. Mixed 15 minutes at 80° C.
2. In a separate vessel, blended Part B ingredients. And heated to 80° C. and mixed well.
3. Added Part A to Part B with good agitation while maintaining emulsion temperature at 80° C.
4. Combined Part C ingredients. And added to emulsion. Mixed continuously while cooling to 40° C.
5 Adjusted pH between 6.0 to 6.5
6. Added Part D (preservative) to emulsion. Mixed well.
7. Cooled and filled.
   Ingredients List:
N-Hance 3215—Molecular weight 1,350,000; Cationic DS 0.19 from Hercules Inc.
Alkamuls® EGMS: Rhodia Corp
Inustrene® 5016: Witco Corporation
Drakeol® 7: Penreco, Pennzoil Products Company
Lipolan 98: Lipo Chemicals
Crodacol® C95: Croda Inc
Natrosol® Plus—Hercules Inc.
Comment on Appearance: Glossy with slightly grainy texture.pH=6.48 RVT/Helipath @ 5 RPM, Spindle #94=91,600 cP.

### EXAMPLE 16

Hand and Body Lotion with Cationic guar of this invention

| Ingredient | | Weight % | Function |
|---|---|---|---|
| A. | Natrosol Plus CS, Grade 330 | 0.50 | Rheology, Co- |
| | | | emulsifier |
| | Cationic guar (ADPP 6486) | 0.25 | conditioner |
| | Distilled water | 78.00 | solvent |
| | Glycerin, | 2.00 | humectant |
| B. | Glycol stearate (Alkamuls EGMS) | 2.75 | emulsifier, opacifier |
| | Stearic acid (Industrene 5016) | 2.50 | surfactant, |
| | | | emulsifier |
| | Mineral oil (Drakeol 7) | 2.00 | emollient |
| | Acetylated lanolin (Lipolan 98) | 0.50 | skin/hair conditioner |
| | Cetyl alcohol (Crodacol C95) | 0.25 | surf., emulsifier, |
| | | | opacifier |
| C. | Distilled water | 10.00 | solvent |
| | Triethanolamine | 0.50 | pH adjust |
| D. | Propylene glycol and diazolidinyl | 0.75 | biocide |
| | urea and methylparaben | | |
| | and propylparaben #GBT-409 | | |
| | | 100.00 | |

Procedure:
1. Natrosol® Plus was dispersed in water by adding to the vortex of well-agitated from Part A. It was mixed for five minutes. Next solution pH was raised between 8 to 8.5 with NaOH and mixed until Natrosol Plus was fully dissolved. Next cationic guar was added and pH was lowered to about 7 to 7.5. Mixed the solution until cationic guar is dissolved. Next, glycerin was added with continued mixing and heated to 80° C. Mixed 15 minutes at 80° C.
2. In a separate vessel, blended Part B ingredients. And heated to 80° C. and mixed well.
3. Added Part A to Part B with good agitation while maintaining emulsion temperature at 80° C.
4. Combined Part C ingredients. And added to emulsion. Mixed continuously while cooling to 40° C.
5 Adjusted pH between 6.0 to 6.5
6. Added Part D (preservative) to emulsion. Mixed well.
7. Cooled and filled.
   Ingredients List:
Cationic guar—ADPP6486, Molecular weight 1,920,000, Cationic DS 2.1 from Hercules Inc.
Alkamuls® EGMS: Rhodia Corp
Inustrene® 5016: Witco Corporation
Drakeol® 7: Penreco, Pennzoil Products Company
Lipolan 98: Lipo Chemicals
Crodacol® C95: Croda Inc
Natrosol® Plus—Hercules Inc.
Comment on Appearance: Glossy, smooth and creamy. pH=6.23
RVT/Helipath @ 5 RPM, Spindle #94=71,600 cP.

### EXAMPLE 17

Hand and Body Lotion without Cationic Guar

| Ingredient | | Weight % | Function |
|---|---|---|---|
| A. | Natrosol Plus CS, Grade 330 | 0.50 | Rheology, Co-emulsifier |
| | Distilled water | 78.25 | solvent |
| | Glycerin, USP | 2.00 | humectant |
| B. | Glycol stearate | 2.75 | emulsifier, opacifier |
| | (Alkamuls EGMS) | | |
| | Stearic acid (Industrene 5016) | 2.50 | surfactant, emulsifier |
| | Mineral oil (Drakeol 7) | 2.00 | emollient |
| | Acetylated lanolin | 0.50 | skin/hair conditioner |
| | (Lipolan 98) | | |
| | Cetyl alcohol (Crodacol C95) | 0.25 | surf., emulsifier, |
| | | | opacifier |
| C. | Distilled water | 10.00 | solvent |
| | Triethanolamine | 0.50 | pH adjust |
| D. | Propylene glycol and | 0.75 | biocide |
| | diazolidinyl urea and | | |
| | Methylparabene and | | |
| | propylparaben | | |
| | | 100.00 | |

Procedure:
1. Natrosol® Plus polymer was dispersed in water by adding to the vortex of well-agitated from Part A. It was mixed for five minutes. Next solution pH was raised between 8 to 8.5 with NaOH and mixed until Natrosol Plus was fully dissolved. Next, pH was lowered to about 7 to 7.5. Mixed the solution until cationic guar is dissolved. Next, glycerin was added with continued mixing and heated to 80° C. Mixed 15 minutes at 80° C.
2. In a separate vessel, blended Part B ingredients. And heated to 80° C. and mixed well.
3. Added Part A to Part B with good agitation while maintaining emulsion temperature at 80° C.
4. Combined Part C ingredients. And added to emulsion. Mixed continuously while cooling to 40° C.
5 Adjusted pH between 6.0 to 6.5
6. Added Part D (preservative) to emulsion. Mixed well.
7. Cooled and filled.
   Ingredients List:
Alkamuls® EGMS: Rhodia Corp
Inustrene® 5016: Witco Corporation
Drakeol® 7: Penreco, Pennzoil Products Company
Lipolan 98: Lipo Chemicals
Crodacol® C95: Croda Inc
Natrosol® Plus—Hercules Inc.
Comment on Appearance: Glossy, smooth and creamy.
pH=6.35
RVT/Helipath @ 5 RPM, Spindle #94=79,200 cP.

While the invention has been described with respect to specific embodiments, it should be understood that the invention should not be limited thereto and that many variations and modifications are possible without departing from the spirit and scope of the invention.

What is claimed:

1. A skin care composition comprising
a) from about 1 to about 90 wt % of a surfactant,
b) a lower limit amount of 0.05 wt % of a cationic polymer wherein
i) the cationic polymer has a weight average molecular weight (Mw) from about 2,000 to about 10,000,000 Dalton, and
ii) the cationic polymer has a cationic degree of substitution (DS) has a lower limit of 0.25 and has a upper limit of 3.0, and
c) at least one skin care active ingredient,
wherein the skin care composition provides protection to the skin.

2. The skin care composition of claim 1, wherein the skin care compositions has significantly improved stability and clarity as compared to similar prior art compositions.

3. The skin care composition of claim 1, wherein protection provides at least one of the functions selected from the group consisting of cleansing, moisturizing, firming, conditioning, occlusive barrier, emolliency, depositing, anti-wrinkling and mixture thereof to the skin.

4. The skin care composition of claim 1, wherein the active skin care ingredient is selected from the group consisting of perfumes, skin coolants, emollients, deodorants, antiperspirants actives, moisturizing agents, cleansing agents, sunscreen actives, shaving actives, beauty aids, exffolient agent, bleaching agent, soothing and healing agent, anti-oxidant agent, antimicrobial agent, conditioning agent, depositing agent, tanning agent, alpha and beta hydroxyl acids, rejunvenating agent, medicaments agent, nail care active, and mixtures thereof.

5. The skin care composition of claim 1 wherein the cationic polymer is present in the composition with the lower limit amount of 0.1 wt %.

6. The skin care composition of claim 1 wherein the cationic polymer is present in the composition with the lower limit amount of 0.5 wt %.

7. The skin care composition of claim 1 wherein the cationic polymer is present in the composition with the lower limit amount of 1.0 wt %.

8. The skin care composition of claim 1 wherein the cationic polymer is present in the composition with the upper limit amount of 10.0 wt %.

9. The skin care composition of claim 1 wherein the cationic polymer is present in the composition with the upper limit amount of 5.0 wt %.

10. The skin care composition of claim 1 wherein the cationic polymer is present in the composition with the upper limit amount of 3.0 wt %.

11. The skin care composition of claim 1 wherein the composition has a cationic degree of substitution (DS) lower limit of 0.4.

12. The skin care composition of claim 1, wherein the cationic degree of substitution (DS) has a lower limit amount of 0.8.

13. The skin care composition of claim 1, wherein the cationic degree of substitution (DS) has an upper limit amount of 2.0.

14. The skin care composition of claim 1, wherein the cationic degree of substitution (DS) has an upper limit amount of 1.0.

15. The skin care composition of claim 1 wherin the cationic polymer is at least one cationic polygalactomannan or cationic derivatized polygalactomannan.

16. The skin care composition of claim 15, wherein the derivative moiety on the cationic derivatized polygalactomannan is selected from the group consisting of alkyl, hydroxyalkyl, alkylhydroxyalkyl, and carboxymethyl wherein the alkyl has a carbon chain containing from 1 to 30 carbons and the hydroxyalkyl is selected from the group consisting of hydroxyethyl, hydroxypropyl, hydroxybutyl and mixture thereof.

17. The skin care composition of claim 15, wherein the polygalactomannan is selected from the group consisting of guar, locust bean, honey locus, and flame tree.

18. The skin care composition of claim 15, wherein the cationic moiety is selected from quaternary ammonium compounds.

19. The skin care composition of claim 18, wherein the quaternary ammonium compound is selected from the group consisting of 3-chloro-2-hydroxypropyltrimethylammonium chloride, 2,3-epoxy-propyltrimethylammonium chloride, 3-chloro-2-hydroxypropyltrimethylammonium bromide, 2,3-epoxy-propyltrimethylammonium bromide; glycidyltrimethylammonium chloride, glycidyltriethylammonium chloride, gylcidyltripropylammonium chloride, glycidylethyldimethylammonium chloride, glycidyidiethylmethylammonium chloride, and their corresponding bromides and iodides; 3-chloro-2-hydroxypropyltrimethylammonium chloride, 3-chloro-2-hydroxypropyltriethylammonium chloride, 3-chloro-2-hydroxypropyltripropylammonium chloride, 3-chloro-2-hydroxypropylethyldimethylammonium chloride, and their corresponding bromides and iodides; and halides of imidazoline ring containing compounds.

20. The skin care composition of claim 1, wherein the Mw has a lower limit of 10,000.

21. The skin care composition of claim 1, wherein the Mw has a lower limit of 50,000.

22. The skin care composition of claim 1, wherein the Mw has a lower limit of 100,000.

23. The skin care composition of claim 1, wherein the Mw has a lower limit of 400,000.

24. The skin care composition of claim 1, wherein the Mw has an upper limit of 5,000,000.

25. The skin care composition of claim 1, wherein the Mw has an upper limit of 2,000,000.

26. The skin care composition of claim 1, wherein the Mw has an upper limit of 1,000,000.

27. The skin care composition of claim 1, further comprising a member selected from the group consisting of colorant, preservative, antioxidant, activity enhancer, emulsifier, functional polymer, viscosifying agent, alcohol, fat or fatty compound, antimicrobial compound, silicone material, hydrocarbon polymer, oil, suspending agents, stabilizing biocide, pH modifier, flavor, fragrane, salt, and mixtures thereof.

28. The skin care composition of claim 27, wherein the functional polymer is selected from the group consisting of anionic, hydrophobically-modified, and amphoteric acrylic acid copolymers, vinylpyrrolidone homopolymers and copolymers, cationic vinylpyrrolidone copolymers, nonionic, cationic, anionic, and amphoteric cellulosic polymers, acrylamide homopolymers, cationic, anionic, amphoteric, and hydrophobically-modified acrylamide copolymer, polyethylene glycol polymer and copolymer, hydrophobically-modified polyether, hydrophobically-modified polyetheracetal, hydrophobically-modified polyetherurethane, an associative polymer, hydrophobically-modified cellulosic polymer, polyethyleneoxide-propylene oxide copolymer, and a nonionic, anionic, hydrophobically-modified, amphoteric, and cationic polysaccharides, chitosan, and mixtures thereof.

29. The skin care composition of claim 28, wherein the nonionic, cationic, anionic, and amphoteric cellulosic polymers are selected from the group consisting of hydroxyethylcellulose, hydroxypropylcellulose, hydroxypropylmethylcellulose, carboxymethylcellulose, hydrophobically-modified carboxymethylcellulose, cationic hydroxyethylcellulose, cationic hydrophobically-modified hydroxyethyl cellulose, hydrophobically modified hydroxyethylcellulose, hydrophobically-modified hydroxypropylcellulose, cationic hydrophobically-modified hydroxypropyl cellulose, cationic carboxymethylhydroxyethylcellulose, and cationic hydroxypropylcellulose.

30. The skin care composition of claim 28, wherein the nonionic, anionic, hydrophobically modified, amphoteric, and cationic polysaccharides are selected from the group consisting of carboxymethyl guar, alginates, hydroxypropyl guar, hydrophobically-modified guar, carboxymethyl guar hydroxypropyltrimethylammonium chloride, guar hydroxypropyltrimethylammonium chloride, and hydroxypropyl guar hydroxypropyltrimethylammonium chloride.

31. The skin care composition of claim 27, wherein the viscosifying agent is selected from the group consisting of NaCl, NH₄Cl, KCl, Na₂SO₄, fatty alcohols, fatty acid esters, fatty acid amides, fatty alcohol polyethyleneglycol ethers, sorbitol polyethyleneglycol ethers, cocamidopropyl betaine, clays, silicas, cellulosic polymers, xanthan, and mixtures thereof.

32. The skin care composition of claim 27, wherein the silicone material is selected from the group consisting of cyclosiloxane, linear siloxane, siloxane structure with polyol, amino, or other functional groups in the siloxane structure, and mixtures thereof.

33. The skin care composition of claim 32, wherein the other functional groups are selected from the group consisting of polyethyleneoxy and/or polypropyleneoxy groups optionally containing C₆-C₂₄ alkyl groups, substituted or unsubstituted amine groups, thiol groups, alkoxylated groups, hydroxyl groups, acyloxyalkyl groups.

34. The skin care composition of claim 27, wherein the silicone material is selected from the group consisting of polyalkylsiloxanes, polyarylsiloxanes, polyalkylarylsiloxanes, and mixtures thereof.

35. The skin care composition of claim 34, wherein the polyalkylsiloxanes are selected from the group consisting of polydimethylsiloxane, polydimethylsiloxane hydroxylated at the end of the chain, and mixtures thereof.

36. The skin care composition of claim 1, wherein the surfactant is an insoluble or soluble surfactant selected from the group consisting of anionic, cationic, amphoteric, zwitterionic nonionic, and mixtures thereof.

37. The skin care composition of claim 36, wherein the surfactant is anionic surfactants selected from the group consisting of alkyl and alkyl ether sulfates sulfonates, sulfosuccinates, sarcosinates, carboxylates isethionates, and mixture thereof.

38. The skin care composition of claim 36, wherein the surfactant is cationic surfactants selected from the group consisting of amino or quaternary ammonium hydrophilic moieties which are positively charged when dissolved in the aqueous composition of the present invention.

39. The skin care composition of claim 36, wherein the surfactant is amphoteric surfactants selected from the group consisting of derivatives of aliphatic secondary and tertiary amines in which the aliphatic radical is a straight or branched chain and wherein one of the aliphatic substituents contains from about 8 to about 18 carbon atoms and one contains an anionic water solubilizing group of carboxy, sulfonate, sulfate, phosphate, phosphonate, or mixture thereof.

40. The skin care composition of claim 36, wherein the surfactant is Zwitterionic surfactants selected from the group consisting of derivatives of aliphatic quaternary ammonium, phosphonium, and sulfonium compounds, in which the aliphatic radicals are straight or branched chain, and wherein one of the aliphatic substituents contains from about 8 to about 18 carbon atoms and one contains as anionic water-solubilizing group of carboxy, sulfonate, sulfate, phosphate, phosphonate, or mixtures thereof.

41. The skin care composition of claim 36, wherein the surfactant is nonionic surfactants selected from the group consisting of compounds containing a hydrophobic moiety and a nonionic hydrophilic moiety, wherein the hydrophobic moiety is selected from the group consisting of alkyl, alkyl aromatic, dialkyl siloxane, polyoxyalkylene, fluoro-substituted alkyls and mixture thereof and the hydrophilic moieties is selected from the group consisting of polyoxyalkylenes, phosphine oxides, sulfoxides, amine oxides, amides, and mixture thereof.

42. The skin care composition of claim 1, wherein the skin care composition is selected from the group consisting of body wash, shower gels, liquid soaps, bar soaps, skin lotions, skin creams, after shower lotions, after cleansing lotions, shave products, after shave products, deodorizing products, antiperspirant products, skin cleansing wipes, skin cooling wipes, skin conditioning wipes, skin drug delivery products, insect repellent products, and sun care products.

43. The skin care composition of claim 4, wherein conditioning agent is selected from the group consisting of silicone materials, hydrocarbon oils, panthenol and derivatives thereof, pantothenic acid and derivatives thereof, and mixtures thereof.

44. The skin care composition of claim 42, wherein the skin care composition is an oil-in-water or water-in-oil emulsion or solution or dispersion or suspension.
